(19) United States
(12) Patent Application Publication
(10) Pub. No.: US 2023/0420074 A1
(43) Pub. Date: Dec. 28, 2023

(54) VARIANT CALLING OF HIGH COVERAGE SAMPLES WITH A RESTRICTED MEMORY

(71) Applicants: ILLUMINA SOFTWARE, INC., San Diego, CA (US); Illumina Cambridge Limited, Cambridge (GB)

(72) Inventors: Adam Michael Birnbaum, La Jolla, CA (US); Fabian Jobst Klötzl, Cambridge (GB); Arun Visvanath, San Diego, CA (US)

(73) Assignees: ILLUMINA SOFTWARE, INC., San Diego, CA (US); Illumina Cambridge Limited, Cambridge (GB)

(21) Appl. No.: 18/213,507

(22) Filed: Jun. 23, 2023

Related U.S. Application Data
(60) Provisional application No. 63/355,541, filed on Jun. 24, 2022.

(51) Int. Cl. G16B 20/20; G16B 30/00

(52) U.S. Cl. CPC G16B 20/20; G16B 30/00

(57) ABSTRACT

Systems, methods, and apparatus are described herein for identifying callable regions and performing variant calling while operating within allocated memory. A sequencing subsystem may comprise a variant caller or variant caller subsystem. The variant caller may include a calling subsystem configured to identify callable regions and may send the callable regions to a downstream genotyping subsystem of the variant caller. The calling subsystem of the variant caller may be configured to detect a callable region of the sequencing data when a depth of the plurality of reads is above a callable region depth threshold. The calling subsystem of the variant caller may monitor memory used by the callable region and, when the memory used exceeds a memory threshold of a total amount of memory allocated, the calling subsystem may split or spill at least a portion of the callable region to operate within the total amount of allocated memory.

### CROSS-REFERENCE TO RELATED APPLICATIONS

This application claims priority to U.S. Provisional Patent Application No. 63/355,541, filed Jun. 24, 2022, the entirety of which is incorporated by reference herein.

### BACKGROUND

In recent years, biotechnology firms and research institutions have improved hardware and software platforms to determine a sequence of nucleotide bases (or whole genome) and identify variant calls for nucleotide bases that differ from reference bases of a reference genome. Sequencing platforms can monitor tens of thousands or more oligonucleotides to detect more accurate nucleotide-base calls from a larger base-call dataset. For instance, a camera in such sequencing platforms can capture images of irradiated fluorescent tags from nucleotide-bases incorporated into to such oligonucleotides. After capturing such images, sequencing platforms send data to a computing device with sequencing-data-analysis software that aligns nucleotide reads to a reference genome. Based on the aligned nucleotide-fragment reads, sequencing platforms can determine nucleotide-base calls for genomic regions and identify variants within a sample's nucleic-acid sequence.

As the amount of sequencing data that is capable of being analyzed continues to grow, challenges are presented when operating sequencing platforms within certain hardware allocations of the computing systems on which the sequencing platforms are being operated. For example, as sequencing data is loaded into memory, the size of the sequencing data may exceed certain memory allocations and cause processing delays or crashes in the sequencing platforms and/or other applications executing on the computing systems. The sequencing platforms may need to operate within such allocations and continue to grow to handle the increases in sequencing data to be analyzed.

### SUMMARY

Systems, methods, and apparatus are described herein for identifying callable regions and performing variant calling while operating within allocated memory. A sequencing subsystem may comprise a secondary analysis subsystem implemented on one or more devices to perform secondary analysis of sequencing data. For example, the sequencing subsystem may comprise a variant caller or variant caller subsystem. The variant caller may include a calling subsystem configured to identify callable regions for being processed to perform variant calling and/or base calling. The calling subsystem may send the callable regions to a downstream genotyping subsystem of the variant caller. The genotyping subsystem may perform variant calling and/or base calling within the callable region.

The calling subsystem of the variant caller may receive sequencing data comprising a plurality of reads of a genome sequence. The calling subsystem of the variant caller may be configured to detect a callable region of the sequencing data when a depth of the plurality of reads is above a callable region depth threshold. The calling subsystem of the variant caller may monitor its memory usage. The memory threshold may be a fixed or dynamic threshold. When the memory used by the calling subsystem of the variant caller exceeds a memory threshold of a total amount of memory allocated to the calling subsystem, the calling subsystem may split the callable region and send a split portion of the callable region to the genotyping subsystem of the variant caller for variant calling based on the split portion. The sending of the split portion of the callable region to the genotyping subsystem increases availability of the memory used by the calling subsystem.

The calling subsystem may analyze the sequencing data when the memory used by the calling subsystem of the variant caller is within the memory threshold of the total amount of memory allocated to the calling subsystem of the variant caller to identify an insertion, a deletion, or other variant or mutation in the sequencing data within a predefined proximity of an identified split. After identifying the variant or mutation in the sequencing data, the calling subsystem may split the callable region outside of the predefined proximity of the variant or mutation. The variant or mutation and/or the predefined proximity of the identified split may be determined based on population data that is accessed by the calling subsystem.

The calling subsystem may analyze buffered sequencing data to identify a location for the splitting of the callable region within the buffered sequencing data. The calling subsystem may identify a portion of the buffered sequencing data having a read depth that is below a splitting threshold and perform the split of the buffered sequencing data at the identified portion having the read depth that is below the splitting threshold. The split portion of the callable region may be the entirety of the callable region that is currently in the memory used by the calling subsystem. In another example, the calling subsystem may maintain, in the memory used by the calling subsystem of the variant caller, a predefined amount of the sequencing data in the first split portion of the callable region, such that a first split portion and a second split portion of the callable region have an overlap in the sequencing data. The overlap may include a predefined number of bases. The overlap may be determined based on population data or user input that is accessed by the calling subsystem. The genotyping subsystem may remove the overlap in the sequencing data between the split portions of the callable region prior to performing variant calling on the callable region.

In another example embodiment, when the memory used by the calling subsystem of the variant caller is within a memory threshold of a total amount of memory allocated to the calling subsystem of the variant caller, the calling subsystem may spill the callable region to disk storage. The spilled callable region may then be streamed back from disk into memory used by the genotyping subsystem for processing. The entirety of the callable region may be spilled to disk, or a portion of the callable region may be spilled to disk while a second portion of the callable region may be maintained in the memory. The genotyping subsystem may analyze the spilled callable region that is streamed back from the disk and discard one or more portions of the spilled callable region from the memory that will not be used for variant calling prior to streaming additional portions of the spilled callable region to the memory. The genotyping subsystem may have a separate allocation of memory and may monitor a second memory threshold associated with the genotyping subsystem to prevent the second memory threshold from being exceeded while streaming the spilled callable region from the disk.

### BRIEF DESCRIPTION OF THE DRAWINGS

FIG. 1A illustrates a schematic diagram of a system environment.

FIG. 1B shows an example of one or more subsystems that may be implemented by a sequencing subsystem for identifying variants or base calls.

FIGS. 2A-2E include graphs illustrating examples for splitting a callable region into genomic regions.

FIG. 3 is a flowchart of a procedure for splitting a callable region into genomic regions based on a memory threshold.

FIG. 4 is a flowchart of a procedure for spilling a callable region to disk based on a memory threshold.

FIG. 5 is a block diagram of an example computing device.

### DETAILED DESCRIPTION

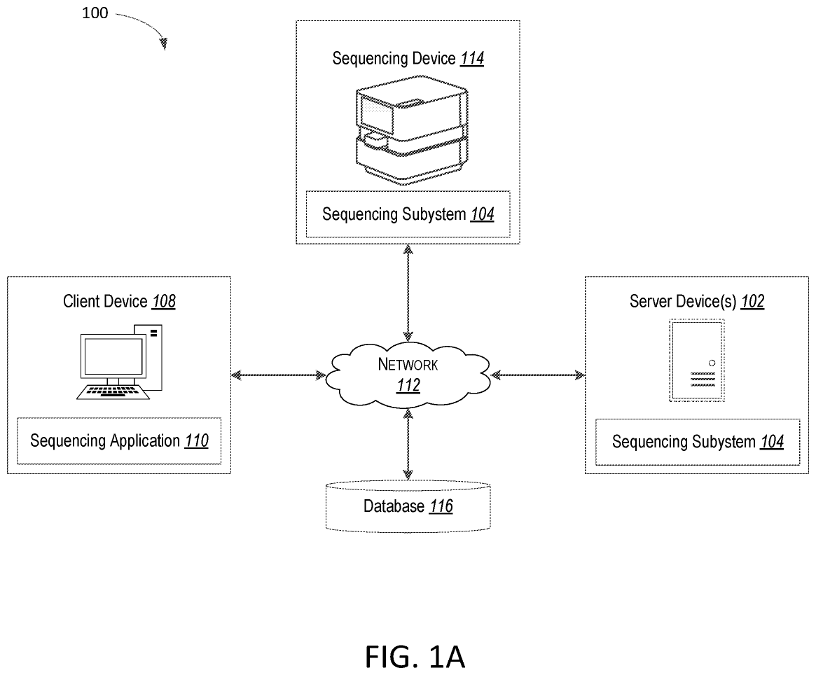

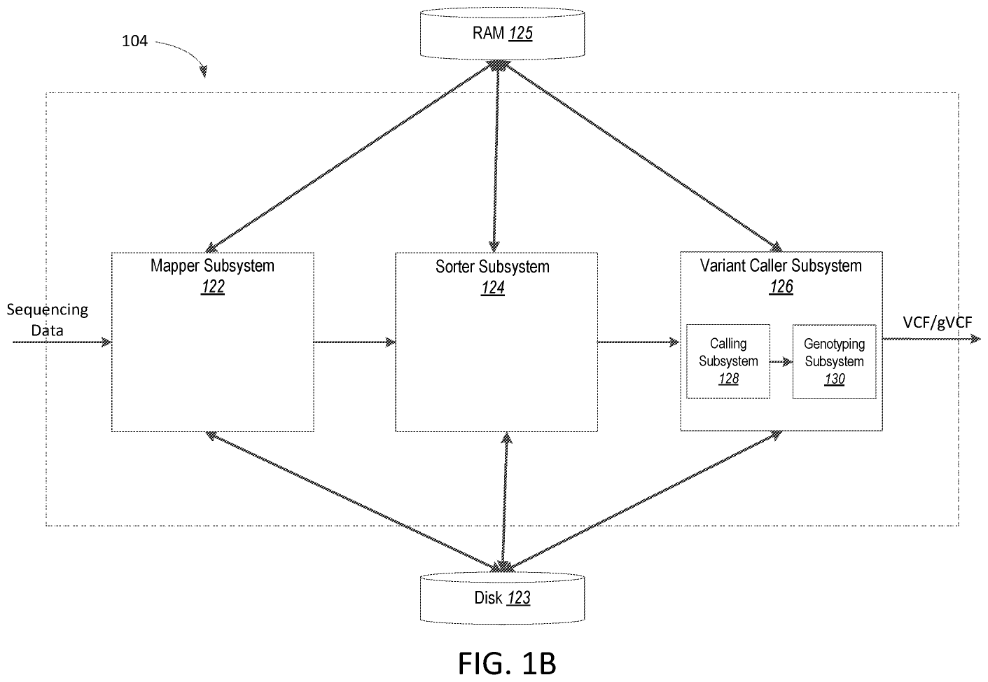

FIG. 1A illustrates a schematic diagram of a system environment (or “environment”) 100, as described herein. As illustrated, the environment 100 includes one or more server device(s) 102 connected to a client device 108 and a sequencing device 114 via a network 112.

As shown in FIG. JA, the server device(s) 102, the client device 108, and the sequencing device 114 may communicate with each other via the network 112. The network 112 may comprise any suitable network over which computing devices can communicate. The network 112 may include a wired and/or wireless communication network. Example wireless communication networks may be comprised of one or more types of radio frequency (RF) communication signals using one or more wireless communication protocols, such as a cellular communication protocol, a wireless local area network (WLAN) or WIFI communication protocol, and/or another wireless communication protocol. In addition, or in the alternative to, communicating across the network 112, the server device(s) 102, the client device 108, and/or the sequencing device 114 may bypass the network 112 and may communicate directly with one another.

As indicated by FIG. 1A, the sequencing device 114 may comprise a device for sequencing a biological sample. The biological sample may include human and non-human deoxyribonucleic acid (DNA) to determine individual nucleotide bases of nucleic-acid sequences (e.g., sequencing by synthesis). The biological sample may include human and non-human ribonucleic acid (RNA). The sequencing device 114 may analyze nucleic-acid segments and/or oligonucleotides extracted from samples to generate nucleotide reads and/or other data utilizing computer implemented methods and systems described herein either directly or indirectly on the sequencing device 114. More particularly, the sequencing device 114 may receive and analyze, within nucleotide-sample slides (e.g., flow cells), nucleic-acid sequences extracted from samples. The sequencing device 114 may utilize sequencing by synthesis (SBS) to sequence nucleic-acid segments into nucleotide reads.

As further indicated by FIG. 1A, the server device(s) 102 may generate, receive, analyze, store, and/or transmit digital data, such as data for determining nucleotide-base calls or sequencing nucleic-acid polymers. As shown in FIG. 1A, the sequencing device 114 may generate and send (and the server device(s) 102 may receive) nucleotide reads and/or other data for being analyzed by the server device(s) 102 for base calling and variant calling. The server device(s) 102 may also communicate with the client device 108. In particular, the server device(s) 102 may send data to the client device 108, including sequencing data or other information and the server device(s) 102 may receive input from the user via client device 108.

The server device(s) 102 may comprise a distributed collection of servers where the server device(s) 102 include a number of server devices distributed across the network 112 and located in the same or different physical locations. Further, the server device(s) 102 may comprise a content server, an application server, a communication server, a web-hosting server, or another type of server.

As further shown in FIG. 1A, the server device(s) 102 and/or the sequencing device 114 may include a sequencing subsystem 104. The sequencing subsystem 104 may be implemented as hardware and/or software on one or more devices to perform secondary analysis of sequencing data. The sequencing subsystem may be implemented as a secondary analysis subsystem on one or more devices to perform secondary analysis. For example, the sequencing subsystem 104 may analyze nucleotide reads and/or other data, such as sequencing metrics received from the sequencing device 114, to determine nucleotide base sequences for nucleic-acid polymers. For example, the sequencing subsystem 104 may receive raw data from the sequencing device 114 and may determine a nucleotide base sequence for a nucleic-acid segment. The raw data may be received from the sequencing device 114 in a file format, such as a FASTQ file, that is capable of being recognized for processing. A FASTQ file may include a text file that contains the sequence data from clusters that pass filter on a flow cell. The FASTQ format is a text-based format for storing both a biological sequence (e.g., such as a nucleotide sequence) and its corresponding quality scores. The sequencing subsystem 104 may process the sequencing data to determine the sequences of nucleotide bases in DNA and/or RNA segments or oligonucleotides.

In addition to processing and determining sequences for biological samples, the sequencing subsystem 104 may generate a file for processing and/or transmitting to other devices. The files that are generated may be in a sequence alignment/map (SAM) format, a binary alignment/map (BAM) format, a compressed reference-oriented alignment map (CRAM) format, and/or another file format for processing and/or transmitting to other devices. The SAM format may be an alignment format for storing reads aligned to a reference genome. The SAM format may support short and long reads (e.g., up to 128 Mb) produced by different sequencing devices 114. The SAM format may be a text format file that is human-readable. The BAM format may maintain the same information in a SAM file, but in a compressed, binary format that is machine-readable. BAM files may show alignments of the reads received in the data received from the sequencing device 114. CRAM files may be stored in a compressed columnar file format for storing biological sequences.

The client device 108 may generate, store, receive, and/or send digital data. In particular, the client device 108 may receive sequencing metrics from the sequencing device 114. Furthermore, the client device 108 may communicate with the server device(s) 102 to receive one or more files comprising nucleotide base calls and/or other metrics. The client device 108 may present or display information pertaining to the nucleotide-base call within a graphical user interface to a user associated with the client device 108.

The client device 108 illustrated in FIG. 1A may comprise various types of client devices. In examples, the client device 108 may include non-mobile devices, such as desktop computers or servers, or other types of client devices. In other examples, the client device 108 may include mobile devices, such as laptops, tablets, mobile telephones, or smartphones.

As further illustrated in FIG. 1A, the client device 108 may include a sequencing application 110. The sequencing application 110 may be a web application or a native application stored and executed on the client device 108 (e.g., a mobile application, desktop application). The sequencing application 110 may include instructions that (when executed) cause the client device 108 to receive data from the sequencing device 114 and present, for display at the client device 108, data to the user of the client device 108, such as data from a variant call file.

As further illustrated in FIG. 1A, the environment 100 may include a database 116. The database 116 can store information such as variant call files, sample nucleotide sequences, nucleotide reads, nucleotide-base calls, sequencing metrics, population data, and/or other data as described herein. The server device(s) 102, the client device 108, and/or the sequencing device 114 may communicate with the database 116 (e.g., via the network 112) to store and/or access information, such as variant call files, sample nucleotide sequences, nucleotide reads, nucleotide-base calls, sequencing metrics, population data, and/or other data as described herein.

The environment 100 may be included in a local network or local high-performance computing (HPC) system. The environment 100 may be included in a cloud computing environment comprising a plurality of server devices, such as server device(s) 102, having software and/or data distributed thereon. The sequencing subsystem 104 may be implemented to operate one or more subsystems as described herein, and may be implemented on a single device, such as a server device 102 or a sequencing device 114, or distributed across multiple devices, such server devices 102 and/or sequencing device 114. The server devices 102 and/or sequencing device 114 may have access to the database 116 via the network 112 in a cloud-based computing system, for example.

Though FIG. 1A illustrates the components of environment 100 communicating via the network 112, it will be appreciated that the components of environment 100 may communicate directly with each other, for example, bypassing the network 112. For example, the client device 108 may communicate directly with the sequencing device 114.

The sequencing subsystem 104 may comprise one or more sequencing subsystems used to analyze the sequencing data received from the sequencing device 114 and/or to perform secondary analysis to identify variants in the sequencing data. The nucleotide-base call may indicate a determination or prediction of the type of nucleotide base that has been incorporated within an oligonucleotide on a nucleotide-sample slide (e.g., read-based nucleotide-base calls) or a determination or prediction of the type of nucleotide base that is present at a genomic coordinate or genomic region within a sample genome. For example, a nucleotide-base call may include a base call corresponding to a genomic coordinate and a reference genome, such as an indication of a variant or a non-variant at a particular location corresponding to the reference genome. A nucleotide-base call may refer to the base that is detected at a position in a read together with a quality score that indicates a confidence in that call. The base call may allow for detection of a mutation or variant based on a comparison between the base call in each read that spans a position and the base that is presented in the reference genome at the same position. The variant may include, but is not limited to, a single nucleotide polymorphism (SNP), an insertion or a deletion (indel), or base call that is part of a structural variant. An insertion changes the DNA sequence by adding one or more nucleotides to the sequence as compared to the reference genome. A deletion changes the DNA sequence by removing at least one nucleotide from the sequence as compared to the reference genome. The deleted DNA may alter the function of the affected protein or proteins. A single nucleotide-base call can comprise an adenine call, a cytosine call, a guanine call, or a thymine call for DNA (abbreviated as A, C, G, T) or a uracil call (instead of a thymine call) for RNA (abbreviated as U). A mutation may include a single change or difference in the genetic sequence. The variant may comprise a sequence that comprises one or more mutations.

FIG. 18 shows an example of one or more subsystems that may be implemented by the sequencing subsystem 104 for identifying variants. As shown in FIG. 1B, the sequencing subsystem 104 may implement a mapper subsystem 122, a sorter subsystem 124, and/or a variant caller subsystem 126. The one or more subsystems may be implemented for performing secondary analysis. The mapper subsystem 122 may be implemented to align the reads in sequencing data received from the sequencing device 114 and/or stored at the server device(s) 102. The reads in the sequencing data produced by the sequencing device 114 and/or generated and stored in the files by the server device(s) 102 may not be included in a single sequence with all DNA information. Instead, the sequencing data produced by the sequencing device 114 and/or generated in the files by the server device(s) 102 may include a number of short subsequences, or reads, with partial DNA information. Read alignment may be performed by the mapper subsystem 122 to map reads to a reference genome and identify the location of each individual read on the reference genome. The mapper subsystem 122 may stream unaligned reads from the sequencing data as FASTQ or ILLUMINA individual base call (BCL) files and perform read alignment on the sequencing data therein. FASTQ files can contain up to millions of entries and can be several megabytes (Mbs) or gigabytes (GBs) in size. The mapper subsystem 122 may output the aligned reads in an aligned BAM file, as described herein.

The BAM files may include a header section and an alignment section. The header section may include information about the file, such as sample name, sample length, and alignment method. The alignment section may include a read name, read sequence, read quality, alignment information, and other custom tags for the read. For each read or read pair, the alignments section may include a read group. The read group may include a subset of reads on a flow cell from the same lane, sample, and/or library prep. Different read groups may have different coverage or different depth. The depth may be determined by a number of reads aligned to a location in the sequence with a certain quality. The depth may be determined by a number of reads aligned to a location in the sequence with a certain quality. The number of reads may be determined for one or more read groups. The alignment section may include a barcode tag that indicates a demultiplexes sample identifier associated with the read. The alignment section may include a single-end alignment quality. The alignment section may include an edit distance tag, which records the Levenshtein distance between the read and the reference.

Read alignment may be performed using a hash table. A hash table may be built for the genome reference, which may enable a sub-portion of the read, or seed, to be mapped to the genome. The location of the read may be determined from the result of seed extension at each of its mapping locations. The mapper subsystem 122 may use a hash table index of a reference genome to map many overlapping seeds from each read to exact matches in the reference. The hash table may be constructed from any chosen reference with a multi-threaded tool, and loaded into random access memory (RAM) 125. For example, the RAM 125 may comprise a field programmable gate army (FPGA)-board dynamic RAM (DRAM) on the server device(s) 102. The hash table may be stored on the RAM 125 prior to mapping operations performed by the mapper subsystem 122. The read-mapping process may be performed by FPGA logic on the RAM 125.

After the read alignment is performed at the mapper subsystem 122, the aligned sequencing data may be passed downstream to the sorter subsystem 124 to sort the reads by reference position, and polymerase chain reaction (PCR) or optical duplicates are optionally flagged. An initial sorting phase may be performed by the sorter subsystem 124 on aligned reads returning from the RAM 125. Final sorting and duplicate marking may commence when mapping completes. The sorter subsystem 124 may write another BAM file that includes sorted sequencing data to RAM 125 for being accessed downstream by the variant caller subsystem 126.

The variant caller subsystem 126 may be used to call variants from the aligned and sorted reads in the sequencing data. For example, the variant caller subsystem may receive the sorted BAM file as input and process the reads to generate variant data to be included in a variant call file (VCF) or a genomic variant call format (gVCF) file as output from the variant caller subsystem 126.

The variant caller subsystem 126 may comprise a calling subsystem 128 and/or a genotyping subsystem 130. As the variant caller subsystem 126 receives the sequencing data, the calling subsystem 128 may identify callable regions with sufficient aligned coverage. The callable regions may be identified based on a read depth. The read depth may represent a number of times a particular base is represented within each of the reads in the sequencing data. Sometimes the wrong base may be incorporated into a DNA fragment identified in the sequencing data. For example, a camera in the sequencing device 114 may pick up the wrong signal, the mapper subsystem 122 may misplace a read, or a sample may be contaminated to cause an incorrect base to be called in the sequencing data. By sequencing each fragment numerous times to produce multiple reads, there is a confidence or likelihood that identified variants are true variants and not artefacts from the sequencing process. The read depth represents the number of times each individual base has been sequenced or the number of reads in which the individual base appears in the sequencing data. The higher the read depth, the greater the level of confidence in variant calling.

The callable regions may be the regions that are passed downstream to the genotyping subsystem 130 for calling variants from the callable region. For example, the genotyping subsystem 130 may compare the callable region to a reference genome for variant calling. The calling subsystem 128 may identify a callable region when the read depth of the sequencing data is above a callable region depth threshold. For example, the calling subsystem 128 may identify a callable region in the sequencing data when the read depth of one or more sequence fragments is above a depth threshold of one. After the callable region is identified, the calling subsystem 128 may pass the callable region to the genotyping subsystem 130, which may turn the callable region into an active region for generating potential positions in the active region where there may be variants. The genotyping subsystem 130 may identify a probability or call score of whether a potential position includes a variant.

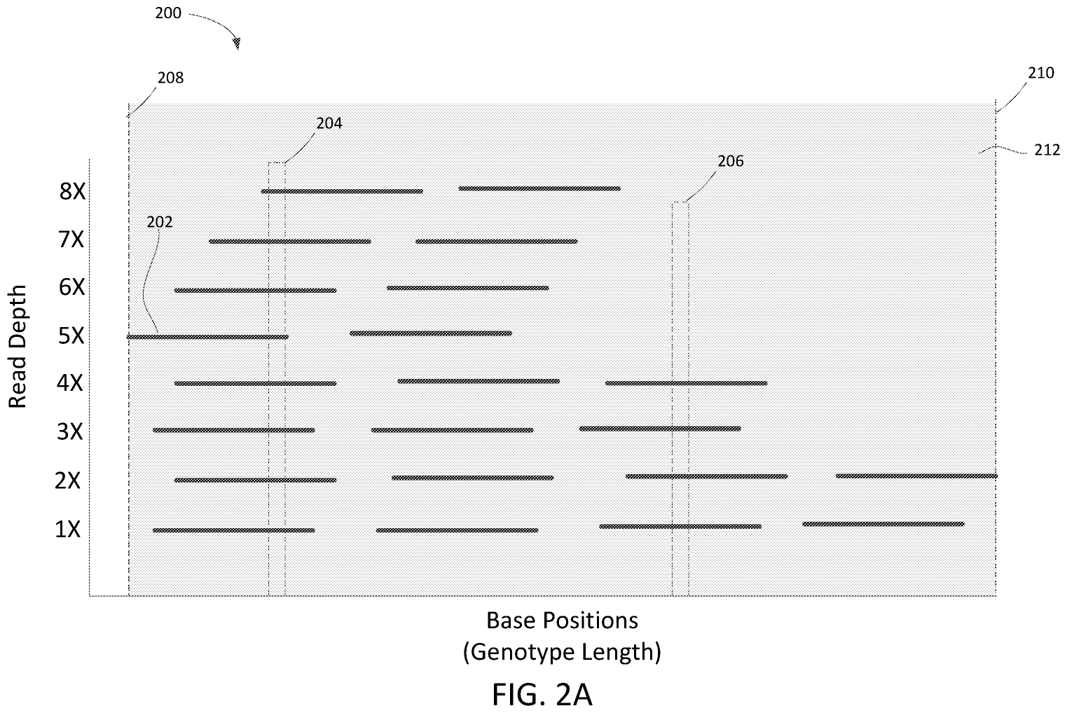

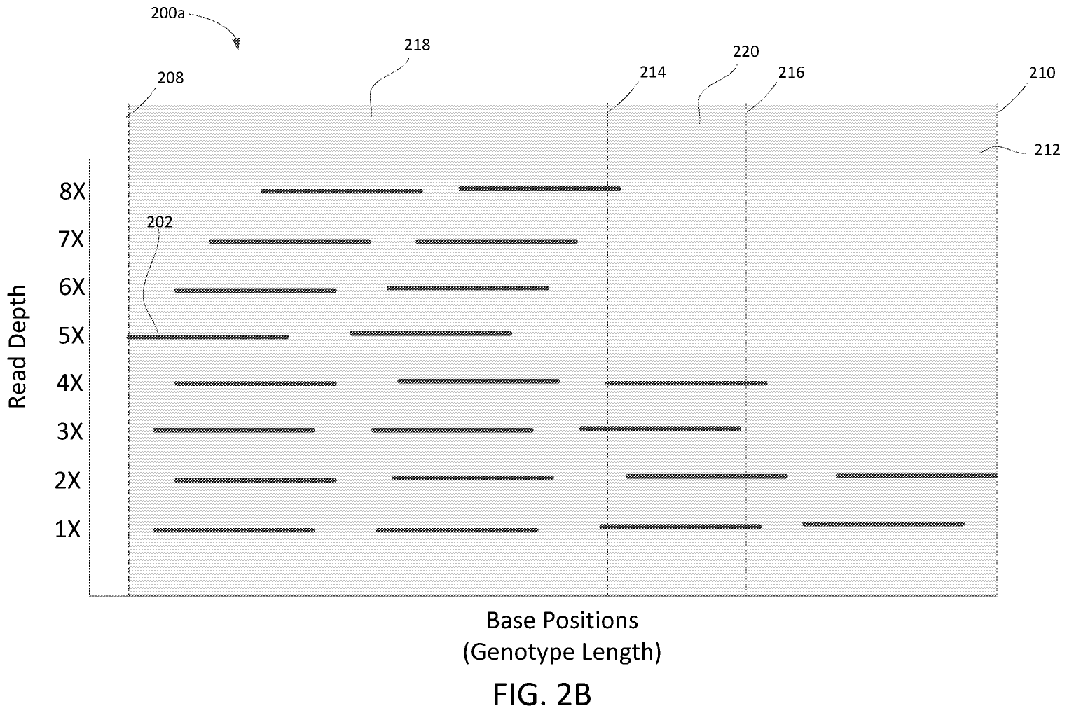

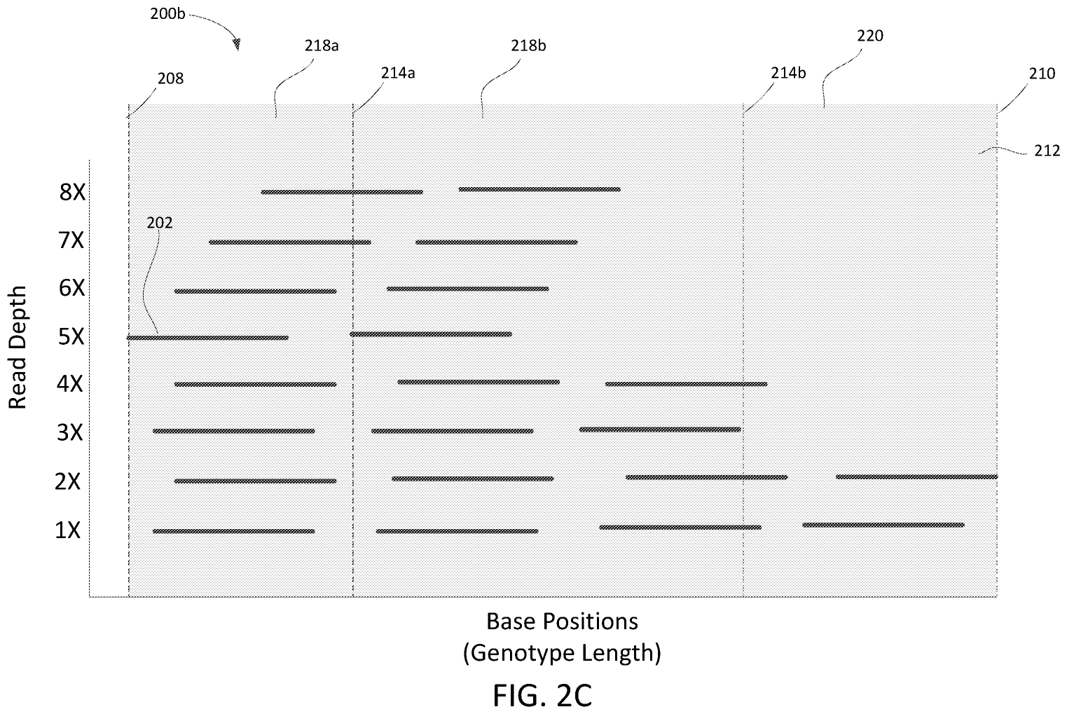

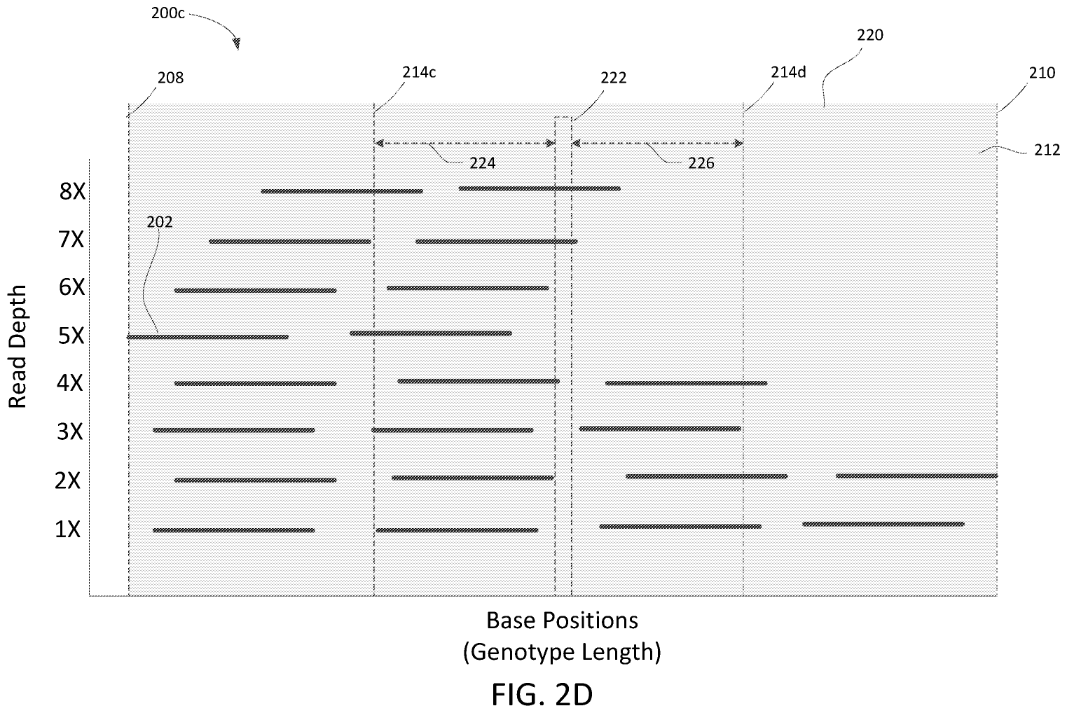

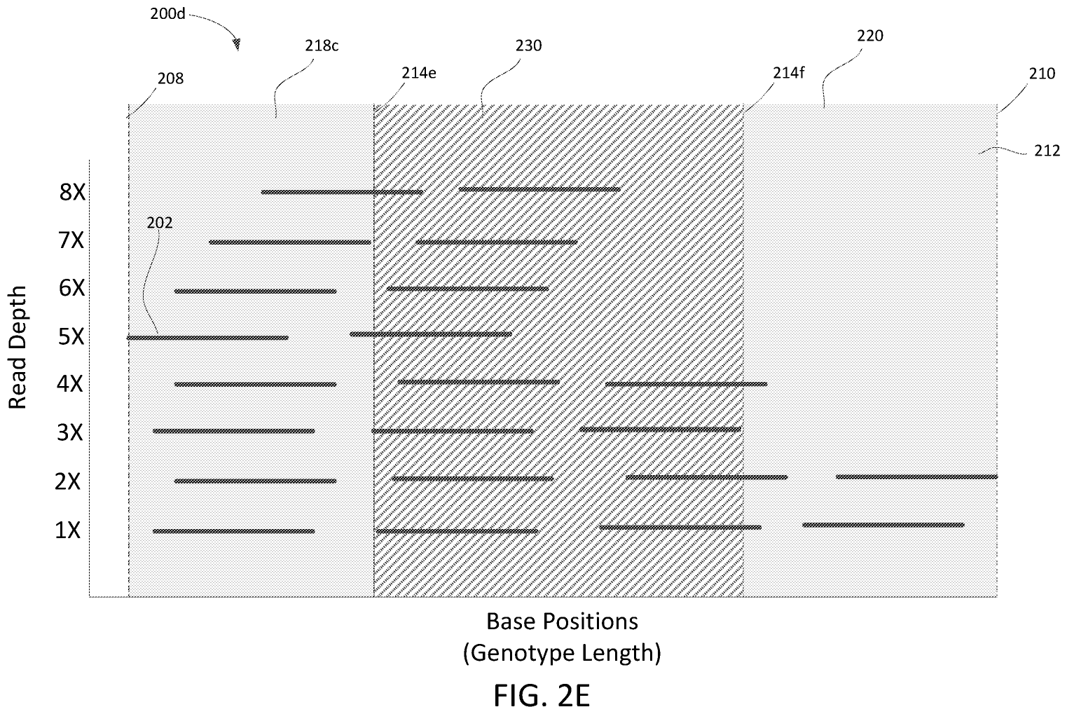

FIG. 2A includes a graph 200 illustrating an example of a callable region based on read depth of genomic regions in the sequencing data. As shown in FIG. 2A, the sequencing data may include a number of short subsequences, or reads 202, with partial DNA information. The reads 202 may be overlapping at a given reference base position, or nucleotide. For example, at a given genomic region 204 in the sequencing data, there may be 8 overlapping reads 202. As such, the genomic region 204 in the sequencing data may have a read depth of 8. At another genomic region 206 in the sequencing data, there may be 4 overlapping reads 202. As such, the genomic region 206 in the sequencing data may have a read depth of 4. The read depth may be the number of the mapped reads at each base position in the sequencing data. The genomic regions 204, 206 may each comprise one or more reference base positions, for example.

As described herein, a callable region 212 may be identified by the calling subsystem of the variant caller based on the read depth of the sequencing data. The calling subsystem of the variant caller may identify a read depth of the callable region 212 reaches or is above a callable region depth threshold at a location 208 in the sequencing data. The callable region depth threshold may be, for example, a read depth of zero or one, such that a callable region 212 may begin when a read depth is detected. However, other depth thresholds may be implemented. The callable region 212 may continue to be buffered in memory (e.g., RAM 125 shown in FIG. 1B) by the calling subsystem for being sent to the genotyping subsystem of the variant caller for detecting the variants in the callable region 212. The calling subsystem may identify that the read depth reaches or falls below the callable region depth threshold at a location 210 in the sequencing data and identify an end of the callable region 212. The callable region 212 may then be sent to or accessed by the genotyping subsystem of the variant caller for detecting the variants in the callable region 212.

Referring again to FIG. 1B, the calling subsystem 128 of the variant caller subsystem 126 may continue to buffer the callable region in RAM 125 so long as the read depth of the callable region is above the callable region depth threshold in the sequencing data. The calling subsystem 128 may attempt to buffer the entire callable region in the RAM 125 so as not to lose sequence context in the sequencing data that may be used for making variant calls in any of the genomic regions of the sequencing data at the genotyping subsystem 130. The sequence context may include one or more reads and/or bases upstream and/or downstream in the sequence from a position on the sequence. The callable region may include a collection of sequences that align to the same area of a reference genome or sequence, which may be referred to as a pileup. A pileup may include a group of reads that overlap the same position having a read depth. The callable region may include a span of reference positions where the sample sequences pile up to a certain depth that aligns with the reference positions. The depth may be indicated by a threshold or value indicating the pile up. For example, a callable region may include a span of reference positions where the sample sequences pile up to a depth greater than a threshold or value. As the sample sequences in a callable region pile up to a greater depth, there may be a greater confidence in variant calling that is performed.

As these callable regions continue to be buffered and/or continue to pile up, they may outgrow the space allocated in RAM 125 to the calling subsystem 128. In one example, the allocated space in RAM 125 for the callable region may be 14 GB, 20 GB, or another level of allocated memory in RAM 125. In an example, the entire memory available for being allocated in RAM 125 may be 40 GB of RAM. The callable region may exceed this allocation and/or other allocations.

The sequencing subsystem 104 may be required to stay within global memory limits in the RAM 125 and/or on a hard disk drive (HDD) or disk 123 when processing sequencing data. The RAM 125 and the disk 123 may be different types of memory or storage. The RAM 125 may be used to store programs and data that the processor on the server device(s) 102 operating the sequencing subsystem 104 may use in real time. The RAM 125 may be volatile and may be erased when the computing device is turned off. The disk 123 may be a permanent storage or non-volatile memory that is used to store user specific data, programs, and files that may be accessed when the computing device is turned on after being turned off. For example, the sequencing subsystem 104, and/or subsystems thereof, may be stored on disk 123 as computer-executable instructions that may be loaded into RAM 125 to operate as described herein. The disk 123 may be a network storage that comprises permanent storage shared on one or more computing devices on a network (e.g., cloud storage system).

To keep the sequencing subsystem 104 within the global memory limits during operation, each subsystem may be allocated or impose its own memory limits within RAM 125 and/or on the disk 123. Exceeding these memory limits may cause the operation of the individual subsystem to operate more slowly or crash, may cause the sequencing subsystem 104 as a whole to operate more slowly or crash, and/or may cause other applications operating on the server device(s) 102 on which the sequencing subsystem 104 is operating to operate more slowly or crash. To prevent these memory limits from being exceeded for a given subsystem, the sequencing subsystem 104 and/or the operating system operating on the one or more server devices 102 may monitor the amount of memory being utilized by a given subsystem and cancel or pause the operation of the subsystem to prevent the memory limits from being exceeded.

These memory limits may be particularly difficult for the calling subsystem 128 of the variant caller subsystem 126. As described herein, as the callable regions being identified by the calling subsystem 128 continue to grow in size, the memory limit allocated to and/or imposed by the calling subsystem 128 in the RAM 125 may be exceeded by the size of a callable region before it is passed to the genotyping subsystem 130. The size of the callable region may grow due to the number of reads in various portions of the callable region. For example, given a genome sequenced to an average depth of 300 a portion of the callable region may occupy a large amount of the memory allocated to the calling subsystem 128 in the RAM 125. Similarly, as the genomic length of the callable region grows, the memory allocated to the calling subsystem 128 in the RAM 125 may similarly be occupied and/or exceeded. Thus, the calling subsystem 128 may be unable to fit the entirety of each callable region in the memory buffer allocated in RAM 125, particularly as the depth of these callable regions continues to increase for genome sequencing analysis. As the callable region reaches the allocated memory (e.g., 20 GB) in the RAM 125, the calling subsystem 128 may continue to search for available memory causing the calling subsystem 128 and/or the variant caller subsystem 126 to stall as the allocated memory for the subsystem has been limited. After a period of time, the operation of the variant caller subsystem 126 may be canceled or paused to free up the memory resources in the RAM 125.

In addition to the RAM 125, each of the subsystems may have access to storage in the disk 123. The disk 123 may include non-volatile memory or permanent storage. Thus, the variant caller subsystem 126 and/or the calling subsystem 128 may spill the callable region to the disk 123. However, each time data goes to or from disk, there is a larger cost in processing resources in writing the data to the disk 123 and reading the data from the disk 123 than in accessing the same data from RAM 125. There may also be a greater processing cost in performing compression/decompression of the data for storage on the disk 123. These processing costs may reduce performance of other subsystems in the sequencing subsystem 104 and/or other applications operating on the server device(s) 102.

In an effort to reduce the processing costs associated with reading and writing to the disk 123, each subsystem in the sequencing subsystem 104 may attempt to operate within the memory allocated to the subsystem. In order to operate within the memory allocated to the calling subsystem 128, the calling subsystem 128 may monitor the memory being used in the RAM 125 for buffering the callable region. When the buffered memory reaches a memory threshold of the total amount of RAM 125 allocated to the calling subsystem 128, the calling subsystem 128 may split the callable region into two or more genomic regions. For example, the memory threshold may be set to a percentage (e.g., 70% or 75%) of the total allocated memory in RAM 125 for the calling subsystem 128. In another example, the memory threshold may be set to a predefined amount of memory (e.g., 1 GB), which may cause the calling subsystem 128 to perform a split at or near each threshold amount of memory. The memory threshold may be fixed or dynamic, as further described herein, to cause the splitting of the callable region at different locations. The split may force an end to the callable region in the RAM 125 even though the callable region is not reduced to the callable region depth threshold. The calling subsystem 128 may scan the reads before and/or after the location in the sequence data at which the memory threshold is reached and move a split earlier or later in an attempt to prevent the loss of a variant (e.g., due to sequence context). After splitting a region, the calling subsystem 128 may pass the split region downstream to the genotyping subsystem 130. In one example, the split region may be passed by sending pointers to the reads of the split portion to the genotyping subsystem 130. The memory (e.g., RAM) may remain occupied with the reads of the split portion for processing by the genotyping subsystem 130. The genotyping subsystem 130 may turn the portion of the callable region into an active region for generating potential positions in the active region where there may be variants. The genotyping subsystem 130 may identify a probability or call score of whether a potential position includes a variant in the active region. After the genotyping subsystem 130 processes the reads in the sequencing data, the memory (e.g., RAM) may be freed for receiving additional sequencing data.

When the buffered memory reaches the memory threshold of the total amount of RAM 125 allocated to the calling subsystem 128, the calling subsystem 128 may send the entirety of the buffered portion of the callable region to the genotyping subsystem 130 to free up the entire buffer. In another example, the calling subsystem 128 may identify another location in the buffered sequencing data to make the split. For example, the calling subsystem 128 may analyze the read depth of the buffered sequencing data to identify a location at which to make the split. The calling subsystem 128 may identify a portion of the sequencing data having a read depth that is below a splitting threshold for making the split. The splitting threshold may be set to a read depth that is greater than the callable region depth threshold. However, the splitting threshold may be set to a read depth that prevents the loss of additional sequence context around the location of the split that may be used by the genotyping subsystem 130 to more accurately predict variants during variant calling.

FIG. 28 includes a graph 200a illustrating an example of a location at which a callable region may be split based on read depth of genomic regions in the sequencing data. The sequencing data shown in the graph 200a in FIG. 2B may be similar to the sequencing data in the graph 200 shown in FIG. 2A. However, the calling subsystem may monitor the buffered memory in RAM and identify that the memory threshold has been reached at a location 216 within the sequencing data. The calling subsystem may then analyze the buffered sequencing data to identify a location 214 at which the read depth is at or below the splitting threshold. The callable region 212 may be split at the location 214 (e.g., when the splitting threshold is set to a read depth of 3) and the split portion 218 may be sent downstream to the genotyping subsystem for variant calling. As the splitting threshold may be met at various locations within the buffered sequencing data, the calling subsystem may identify the location 214 that will clear the largest amount of data from the buffer. The remaining portion 220 of the callable region 212 may be left in the buffer and the calling subsystem may continue buffering the sequence data until the callable region depth threshold or another memory threshold is reached.

The calling subsystem may use additional logic that may enable the calling subsystem to make more intelligent splits in a callable region within the sequencing data. For example, the calling subsystem may identify that the memory threshold has been reached, or the sequencing data is within a predefined amount of occupied memory from reaching the memory threshold, and scan the buffered sequencing data to identify a location at which to make a split to prevent the loss of a variant at the genotyping subsystem. The calling subsystem may implement a dynamic memory threshold and/or a dynamic splitting threshold. The dynamic thresholds may allow for more intelligent splits based on the amount of memory occupied in the buffer and the read depth of the sequencing data.

FIG. 2C includes a graph 200b illustrating another example of locations for splitting a callable region based on read depth of genomic regions in the sequencing data. The sequencing data shown in the graph 200b in FIG. 2C may be similar to the sequencing data in the graph 200 and graph 200a shown in FIGS. 2A and 2B, respectively. As shown in FIG. 2C, the calling subsystem may analyze the buffered sequencing data to identify one or more locations 214a, 214b for splitting the callable region 212. For example, the calling subsystem may split the callable region 212 into one or more genomic regions based on a predefined low-end splitting threshold. The calling subsystem may split the callable region 212 each time the low-end splitting threshold is met or exceeded. The low-end splitting threshold may be higher than the callable region depth threshold. As shown in FIG. 2C, the low-end splitting threshold may be set to a depth threshold of 3, which may cause the calling subsystem to split the callable region 212 each time the splitting threshold is met or exceeded in the sequencing data. The predefined low-end splitting threshold may prevent loss of sequence context by splitting the callable region 212 at a location in the sequencing data at which the read depth is relatively lower than other regions in the sequencing data, so there is a loss of less data when performing variant calling at the genotyping subsystem and a lower confidence level of the variant at the split due to the lower depth than if the split occurred at a location in the sequencing data having a higher depth. To avoid the loss of sequence context and/or performing multiple splits within a genomic region that does not occupy at least a minimum level of the available buffer for storage, the calling subsystem may set a low-end memory threshold for making a split. For example, the split at the location 214a may be performed after the low-end memory threshold has been met or exceeded in the buffer. After the split at 214a, the split portion 218a may be sent to the genotyping subsystem for identifying variants.

The low-end splitting threshold and/or the low-end memory threshold may be user configured and/or dynamically updated during the identification of the callable region 212. For example, the low-end splitting threshold and/or the low-end memory threshold may be adjusted based on user input (e.g., received from the sequencing application 110 executing on the client device 108 shown in FIG. 1A). The low-end splitting threshold may also, or alternatively, be dynamically updated based on the amount of available memory left in the buffer. For example, the low-end splitting threshold may increase to predefined read depths as the amount of space occupied in be buffer by the callable region 212 increases. As shown in FIG. 2C, the calling subsystem may determine to split the callable region 212 at a location 214b of the sequencing data that has a higher read depth than the location 214a. After the split at 214b, the split portion 218b may be sent to the genotyping subsystem for identifying variants.

The calling subsystem may include at least a portion of the logic within the genotyping subsystem that is used for identifying variants to prevent the loss of a variant, or loss of context to improve the ability to predict a variant, at the genotyping subsystem. For example, the calling subsystem may identify insertions, deletions, or other variants or mutations within a portion of the sequencing data and prevent splitting of the callable region within a predefined region of the indel.

FIG. 2D includes a graph 200c illustrating another example of locations for splitting a callable region into genomic regions in the sequencing data. The sequencing data shown in the graph 200c in FIG. 2D may be similar to the sequencing data in the graphs 200, 200a, and/or 200b shown in FIGS. 2A, 28, and 2C respectively. As shown in FIG. 2D, the calling subsystem may detect that a memory threshold and/or a splitting threshold has been reached for the callable region 212 and identify a location 214d for performing a split in the callable region 212. For example, the calling subsystem may identify the location 214d based on the read depth of the sequencing data at the location 214d. The calling subsystem may then analyze the buffered sequencing data to determine whether there is an insertion, a deletion, or other variant or mutation within a predefined proximity (e.g., genomic region 226) of the identified splitting location 214d. The genomic region 226 may be a predefined number of bases (e.g., 100 bases, 1,000 bases, etc.), a predefined number of buffer locations, and/or a predefined amount of memory. For example, the calling subsystem may analyze each read 202 and compare the read 202 with the reference sequence to detect the variant or mutation at a genomic portion 222 of one or more of the reads 202. The genomic portion 222 may include one or more bases itself. The bases within the genomic portion 222 may be identified at a starting base or starting index and/or an ending base or an ending index.

The calling subsystem may utilize population data to identify variant or mutation in the genomic portion 222 that are more likely for (or more common to) a population corresponding to the sample genome. Thus, the calling subsystem may utilize various frequency and/or population data that denotes a likelihood of an insertion, a deletion, structural variants, copy number variants, or other variant or mutation occurring within the genomic region 222 and/or the size of the genomic region 222. The calling subsystem may access a database that includes population data. The database may indicate genomic sequences and corresponding genomic coordinates for a given population or ethnic group. The database may also include metadata indicating surrounding nucleotide bases common to the population or ethnic group. The calling subsystem may determine and utilize the population data corresponding to a sample genome. To illustrate, in some embodiments, the calling subsystem may identify or receive data regarding a population and/or ethnic group corresponding to a particular sample genome. Accordingly, the calling subsystem may identify variants or mutations from the bases or genomic portions common for the population or ethnic group. To illustrate, in one or more embodiments, the calling subsystem may utilize a reference genome corresponding to the identified population or ethnic group corresponding to the sample genome. The calling subsystem may identify genomic regions comprising insertions, deletions, structural variants, copy number variants, or other variant or mutation or other variants or mutations at the genomic coordinates within the genomic region, such as the genomic region 222. The calling subsystem may also identify the size of the genomic region 222 based on the reference genome corresponding to the identified population or ethnic group.

The calling subsystem may utilize the data regarding the population and/or ethnic group to identify an insertion, a deletion, structural variant, copy number variant, or other variant or mutation at genomic coordinates or target genomic regions of a sample genome. To illustrate, the calling subsystem may identify a variant or mutation within the genomic region 222 based on surrounding nucleotide bases within a genomic region. Based on the nucleotide bases within a genomic region, the calling subsystem may identify a likelihood of an insertion, a deletion, structural variant, copy number variant, or other variant or mutation for the genomic coordinate or region.

When the calling subsystem detects the variant or mutation at the genomic portion 222 is within the predefined genomic region 226 of the identified splitting location 214d, the calling subsystem may prevent the split from occurring at the location 214d. The calling subsystem may identify another location 214c for performing the split within the buffered portion of the callable region 212. The location 214c may be at least a predefined genomic region 224 from the genomic portion 222 (e.g., from a starting index or starting base of the genomic portion 222). The genomic region 224 may be a predefined number of bases (e.g., 100 bases, 1,000 bases, etc.), a predefined number of buffer locations, and/or a predefined amount of memory. The genomic region 224 may be the same or different from the genomic region 226. The splitting of the calling region 212 at the location 214c that is at least the predefined genomic region 224 may prevent the loss of sequence context at or near the variant or mutation for variant calling at the genotyping subsystem.

The genomic regions 224, 226 may change dynamically or otherwise change based on the type of sequencing data being analyzed. For example, the genomic regions 224, 226 may be determined based on the population data. The calling subsystem may identify a number of bases common for being used as sequencing context for an insertion, a deletion, structural variant, copy number variant, or other variant or mutation based on the population data. The calling subsystem may update the genomic regions 224, 226 based on the number of bases indicated in the population data. The variants or mutations may not be spread equally across a genome. Using the population data, positions may be conserved (e.g., rarely having a mutation) and the split may be performed at these positions in an attempt to avoid splitting within the genomic regions 224, 226 of a variant or mutation.

The calling subsystem may attempt to identify the location 214c having a predefined read depth of the splitting threshold from the genomic portion 222 for identifying another split. If the calling subsystem fails to find a location in the sequencing data having a predefined read depth of the splitting threshold, the splitting threshold may be dynamically updated to allow for a higher or lower read depth, or the calling subsystem may fail to perform another split between the location 208 at the start of the callable region 212 and the genomic portion 222 comprising the variant or mutation. The calling subsystem may then look to split the callable region 212 after the genomic portion 222 comprising the variant or mutation. The calling subsystem may attempt to split the callable region 212 at a location that is outside of the genomic region 224 from the genomic portion 222. The memory threshold may have to be increased to accommodate the split while maintaining the context around the variant or mutation.

As described herein, the splitting of the callable region 212 may cause a loss of sequencing context that may be utilized by the genotyping subsystem for making accurate variant calls. In order to preserve the sequencing context and to prevent other loss of sequencing data when splitting the callable region 212, the calling subsystem may maintain an overlap of data from the callable region 212 in the buffer and send multiple split portions of the callable region to the genotyping subsystem that include the overlapping data. The genotyping subsystem may identify the overlapping data and discard any duplicate data. However, the overlapping data may help ensure that the sequencing context and/or other data around a split is preserved for variant calling.

FIG. 2E includes a graph 200d illustrating another example of locations for splitting a callable region into genomic regions in the sequencing data. The sequencing data shown in the graph 200d in FIG. 2E may be similar to the sequencing data in the graphs 200, 200a, 200b, and/or 200c shown in FIGS. 2A, 2B, 2C, and 2D respectively. As shown in FIG. 2E, the calling subsystem may detect that a memory threshold and/or a splitting threshold has been reached for the callable region 212 and identify a location 214f for performing a split in the callable region 212. For example, the calling subsystem may identify the location 214f based on the read depth of the sequencing data at the location 214f. The calling subsystem may send the split portion 218c to the genotyping subsystem for identifying variants. The split portion 218c may include the sequencing data from the location 214f of the split and the beginning of the callable region 212 or a location of a previous split.

The calling subsystem may maintain an overlapping portion 230 of the sequencing data in memory after sending the split portion 218c to the genotyping subsystem. The overlapping portion 230 may be a predefined portion of the sequencing data from the location 214f to a location 214e. The predefined portion may be a predefined number of bases (e.g., 100 bases, 1,000 bases, etc.), a predefined number of buffer locations, and/or a predefined amount of memory. The predefined number of bases, number of buffer locations, and/or the amount of memory may be predefined and/or user-defined based on user input. The overlapping portion 230 may change dynamically. For example, the overlapping portion 230 may change based on population data. The overlapping portion 230 may be defined by read depth. For example, the calling subsystem may identify the location 214e for defining the overlapping portion 230 of the sequencing data from the location 214f of the split based on a predefined read depth at the location 214e. The genotyping subsystem may maintain the pointers or update the pointers to the reads 202 in the overlapping portion 230 such that the memory of the calling subsystem 128 continues to be occupied by the reads 202 in the overlapping portion 230. When the calling subsystem identifies a subsequent location for splitting the callable region 212, or reaches the location 210 at the end of the callable region 212, the calling subsystem may send the remaining portion 220 of the callable region 212 to the genotyping subsystem for variant calling. The calling subsystem may analyze the split portion 218c and determine not to maintain an overlap based on the data in the split portion 218c. The genotyping subsystem may identify the overlapping portion and remove the duplicate bases prior to performing variant calling. The overlapping data maintained near the split may prevent the loss of data at or near the split.

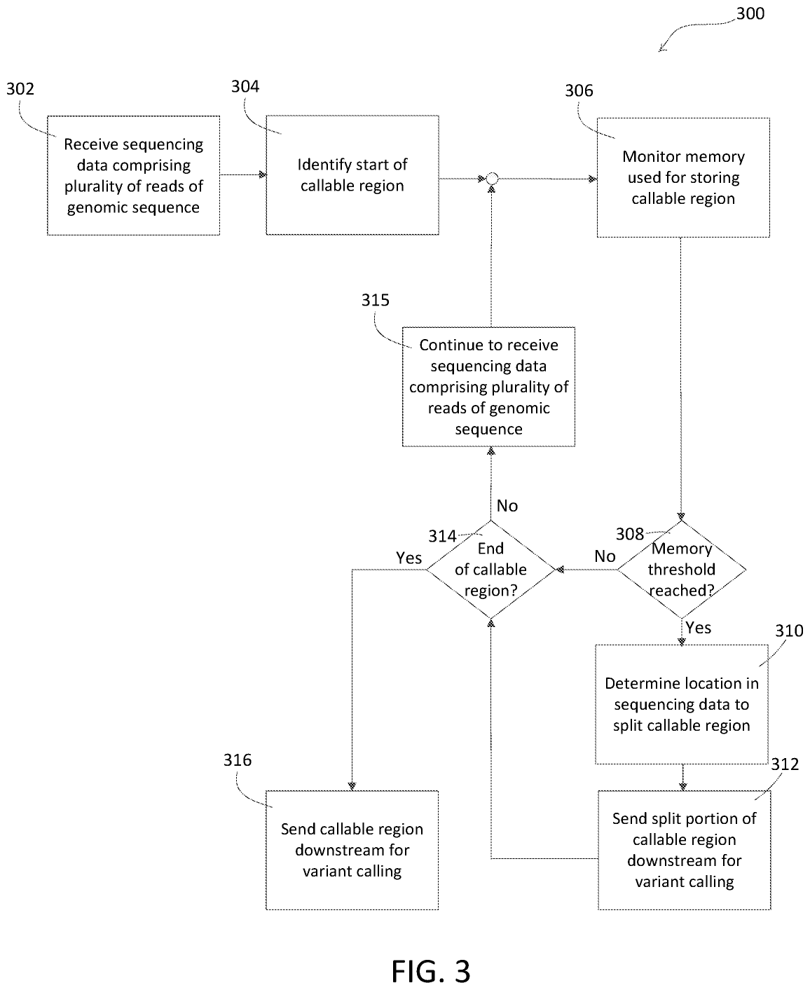

FIG. 3 is a flow diagram of a procedure 300 for splitting a callable region into genomic regions in sequencing data based on a memory threshold. The procedure 300 may be performed by one or more computing devices. For example, the procedure 300 may be performed by one or more server device(s) (e.g., server device(s) 102 shown in FIGS. 1A) and/or sequencing devices (e.g., sequencing device(s) 114) executing one or more portions of a sequencing subsystem (e.g., sequencing subsystem 104 shown in FIGS. 1A and 1B). The procedure 300 may be performed by one or more subsystems of the sequencing subsystem. For example, the procedure 300 may be performed by a calling subsystem (e.g., the calling subsystem 128 shown in FIG. 1B). Though the calling subsystem may be provided as an example subsystem for performing the procedure 300, one or more portions of the procedure 300 may be performed by one or more systems and/or subsystems, such as the sequencing subsystem and/or one or more subsystems therein. The procedure 300 may be performed on a single computing device or may be distributed across multiple computing devices. For example, the procedure 300 may be executed as computer-executable instructions retrieved from memory by one or more processors on one or more computing devices.

As shown in FIG. 3, at 302, the calling subsystem may receive sequencing data comprising a plurality of reads of a genomic sequence. At 304, the calling subsystem may identify a start of a callable region in the sequencing data. For example, the callable region may be identified at the beginning of the sequence data and/or when the sequencing data reaches a callable region depth threshold. The callable region depth threshold may be a value of zero or one. The callable region depth threshold may be predetermined or dynamic, as described herein. The callable region depth threshold for the start of the callable region may be the same or different than the callable region depth threshold for an end of the callable region.

At 306, the calling subsystem may monitor memory used for storing the sequencing data in the callable region. The callable region may be buffered in RAM by the calling subsystem in an attempt to identify the callable region in RAM and send the callable region to the genotyping subsystem for variant calling within the callable region. The memory threshold may be predetermined or dynamic, as described herein. For example, the memory threshold may be set to a relatively larger portion (e.g., bytes, percentage, etc.) of the total allocated memory (e.g., 70%, 75%, etc.) for storing the callable region of the sequencing data to send relatively larger portions of the callable region to the genotyping subsystem, or a relatively smaller portion (e.g., bytes, percentage, etc.) of the total allocated memory (e.g., 25%, 30%, etc.) for storing the callable region of the sequencing data to send relatively smaller portions of the callable region to the genotyping subsystem.

At 308, the calling subsystem may determine whether the memory being used to store the callable region has reached the memory threshold of the allocated memory for storing the callable region. If the memory threshold has not been reached, the calling subsystem may monitor the sequencing data to determine whether an end of the callable region has been reached, at 314. For example, the calling subsystem may identify an end of the callable region in the sequencing data when the sequencing data reaches the callable region depth threshold. For example, the callable region depth threshold may be a value of zero or one. When the end of the callable region is identified, the calling subsystem may send the callable region downstream, at 316, to the genotyping subsystem for variant calling. If the end of the callable region has not been reached, the calling subsystem may continue to receive sequencing data at 315 and continue monitoring the memory used for storing the callable region 306 and/or monitoring for an end of the callable region.

If the calling subsystem determines, at 308, that the memory threshold has been reached, then the calling subsystem may determine a location in the sequencing data to split the callable region at 310. The callable region may be split to preserve the memory resources allocated to the calling subsystem, the sequencing subsystem, and/or the computing device on which the calling subsystem may be operating. The calling subsystem may determine, at 310, to split the callable region at the location in the sequencing data at which the memory threshold has been reached, such that the calling subsystem sends the entirety of the buffered sequencing data comprising the portion of the callable region to the genotyping subsystem. As described herein, the callable region may be split by analyzing the buffered portion of the callable region to identifying another location to perform a split. For example, the split may be performed in a location to reduce an amount of sequencing context that may be lost; avoid a loss of a variant; avoid splitting within a predefined region of a variant and/or mutation, such as an insertion, a deletion, a structural variant, a copy number variant, or another variant or mutation; and/or otherwise preserve data when performing the split.

The location of the split may be determined, at 310, based on a splitting threshold. The splitting threshold may be set to a read depth that is higher than the callable region depth threshold. As described herein, the calling subsystem may analyze the region at which a split may be performed to determine whether to split the callable region in another location. For example, the calling subsystem may identify insertions, deletions, or other variants or mutations within a predefined proximity of the location of the split and identify another location for performing the split. The location of the split may be performed to preserve sequence context data that may be used by the genotyping subsystem to identify variants. The location of the split may be performed based on population data, as described herein.

The split portion of the callable region may be sent downstream, at 312, to the genotyping subsystem for variant calling. The genotyping subsystem may begin analyzing the split portion of the callable region for identifying variants. The calling subsystem may maintain a portion of the split portion of the callable region in the buffer, such that an overlapping portion of two sequential portions of the callable region have an overlap when they are received by the genotyping subsystem. The genotyping subsystem may identify the overlapping portion and remove the duplicate bases prior to performing variant calling. The overlapping data maintained near the split may prevent the loss of data at or near the split. If the location at which the split was made was not the end of the callable region, the calling subsystem may continue receiving sequencing data at 315 and monitoring the memory used for storing the callable region 306 and/or monitoring for an end of the callable region.

Embodiments are described herein for splitting a callable region into one or more portions to operate within memory allocations in RAM for storing the callable region. Alternatively, or additionally, the callable region, or a portion thereof, may be spilled to disk to operate within the memory allocations in RAM. The disk may include non-volatile memory or permanent storage. To avoid incorrect variant calls and preserve the sequence context for making variant calls at the genotyping subsystem, portions of a callable region may be spilled to disk by the calling subsystem and may be streamed back from disk by genotyping subsystem of the variant caller for further analysis. For example, if the callable region reaches a spilling threshold, which may be a memory threshold or a threshold number of bases, the callable region may be spilled to disk. The callable region may be spilled to disk to avoid a risk of losing variant calls at a location of a split. The reads in the callable region may be written to disk to preserve the callable region.

Referring again to FIG. 1B, the calling subsystem 128 of the variant caller subsystem 126 may receive the aligned and sorted sequencing data and may identify callable regions with sufficient aligned coverage. The callable regions may be identified based on a read depth, as described herein. The callable regions may be the regions that are passed downstream to the genotyping subsystem 130 for calling variants from the callable region. For example, the genotyping subsystem 130 may compare the callable region to a reference genome for calling the variants.

The calling subsystem 128 may continue to buffer the callable region in RAM 125 so long as the read depth of the callable region is above the callable region depth threshold in the sequencing data. In order to operate within the memory allocated to the calling subsystem 128, the calling subsystem 128 may monitor the memory being used in the RAM 125 for buffering the callable region. When the buffered memory reaches a memory threshold of the total amount of RAM 125 allocated to the calling subsystem 128, the calling subsystem 128 may spill the callable region to the disk 123 (e.g., HDD). The memory threshold may be the same or different than that of the memory threshold described herein for splitting the callable region.

The calling subsystem 128 may spill the entirety of the callable region to the disk 123 and store as a file that is accessed by the downstream genotyping subsystem 130 when performing variant calling. The calling subsystem 128 may call a function in the genotyping region giving the calling subsystem 128 the beginning and end pointers of the callable region and instructing the calling subsystem 128 to analyze the reads in the callable region. When parts of the callable region are spilled to disk, the calling subsystem 128 may pass information on how to access the reads stored to disk (e.g., using file names or other identifiers). The genotyping subsystem 130 may load the entirety of the callable region into the RAM 125 or stream portions of the spilled callable region back to the RAM 125 from the disk 123. In another example, the calling subsystem 128 may maintain the portion of the callable region in the RAM 125 that is buffered prior to the memory threshold being reached and spill the rest of the callable region to disk. The calling subsystem 128 may update the pointers in the RAM 125 to send the portion of the callable region in the RAM 125 to the genotyping subsystem 130. The genotyping subsystem 130 may begin processing the portion of the callable region received in the RAM 125 first and then stream portions of the callable region from the disk 123 into RAM 125 as the RAM 125 that is allocated to the genotyping subsystem is freed up. As the genotyping subsystem 130 may be allocated a larger portion of the RAM 125 than the calling subsystem, the genotyping subsystem 130 may be able to load a larger portion of the callable region into RAM 125 when processing the callable region.

The genotyping subsystem 130 may stream portions of the spilled callable region from the disk 123 into the RAM 125 and perform an initial analysis to determine whether to maintain the sequencing data in the streamed portion in the RAM 125 prior to accessing additional portions of the spilled callable region from the disk 123. For example, the genotyping subsystem 130 may stream a first portion (e.g., 1 GB, 2 GB, etc.) of the reads in the callable region from the disk 123 and perform an initial analysis to determine whether this first portion of the reads include an insertion, a deletion, or another variant or mutation. The genotyping subsystem 130 may discard the portions of the reads in the callable region in the RAM 125 that will not be used for variant calling (e.g., variants or sequence context for variant calling) and stream a second portion of the callable region from the disk 123. The genotyping subsystem 130 may continue to stream portions of the callable region from the disk 123 to load the portions of the callable region into the RAM 125 that will be used for variant calling. The genotyping subsystem 130 may continue monitoring the amount of the callable region that is loaded into the RAM 125 and use a memory threshold of the amount of memory allocated to the genotyping subsystem 130 to limit the amount of the callable region that is streamed from the disk 123 prior to processing the callable region to free up portion of the RAM 125 prior to uploading additional portions of the callable region. The memory threshold may be a separate memory threshold that is a predefined amount of data less than the allocated memory for the genotyping subsystem 130.

The spilling of the callable region to the disk 123 may allow the calling subsystem 128 to operate within the memory allocations for the callable region, without losing sequence context that may be used for performing variant calling. Maintaining a portion of the callable region in the RAM 125 may also save on processing/compression that may be used to write/read to/from the disk 123 by sending less data to the disk 123.

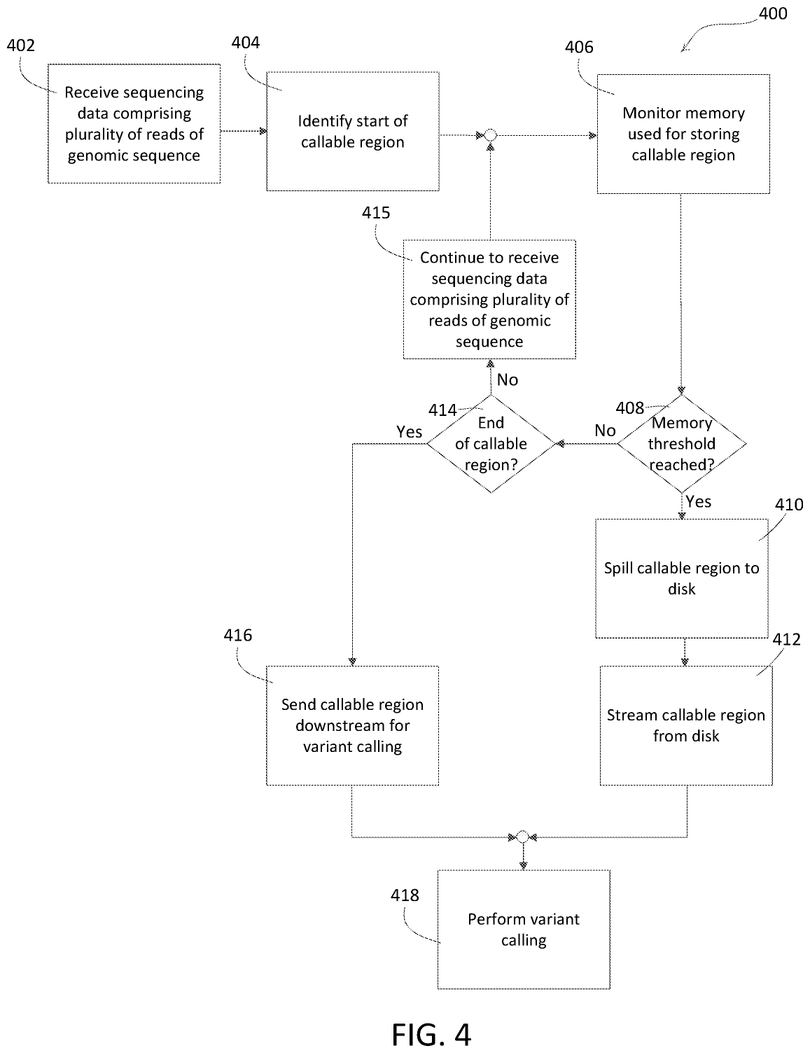

FIG. 4 is a flow diagram of a procedure 400 for spilling a callable region to disk based on a memory threshold. The procedure 400 may be performed by one or more computing devices. For example, the procedure 400 may be performed by one or more server device(s) (e.g., server device(s) 102 shown in FIGS. 1A) and/or sequencing devices (e.g., sequencing device(s) 114) executing one or more portions of a sequencing subsystem (e.g., sequencing subsystem 104 shown in FIGS. 1A and 1B). The procedure 400 may be performed by one or more subsystems of the sequencing subsystem. For example, the procedure 400 may be performed by a calling subsystem and/or a genotyping subsystem (e.g., the calling subsystem 128 and/or the genotyping subsystem 130 shown in FIG. 1B). Though the calling subsystem and the genotyping subsystem may be provided as example subsystems for performing portions of the procedure 400, one or more portions of the procedure 400 may be performed by one or more systems and/or subsystems, such as the sequencing subsystem and/or one or more subsystems therein. Additionally, though one of the calling subsystem or the genotyping subsystem may be described as performing one or more features herein, the features may be performed by the other subsystem. The procedure 400 may be performed on a single computing device or may be distributed across multiple computing devices. For example, the procedure 400 may be executed as computer-executable instructions retrieved from memory by one or more processors on one or more computing devices.

As shown in FIG. 4, at 402, the calling subsystem may receive sequencing data comprising a plurality of reads of a genomic sequence. At 404, the calling subsystem may identify a start of a callable region in the sequencing data. For example, the callable region may be identified at the beginning of the sequence data and/or when the sequencing data reaches a callable region depth threshold.

At 406, the calling subsystem may monitor memory used for storing the sequencing data in the callable region. The callable region may be buffered in RAM by the calling subsystem in an attempt to identify the callable region in RAM and send the callable region to the genotyping subsystem for variant calling within the callable region. The memory threshold may be set to a portion (e.g., bytes, percentage, etc.) of the total allocated memory (e.g., 70%, 75%, etc.) for storing the callable region of the sequencing data.

At 408, the calling subsystem may determine whether the memory being used to store the callable region has reached the memory threshold of the allocated memory for storing the callable region. If the memory threshold has not been reached, the calling subsystem may monitor the sequencing data to determine whether an end of the callable region has been reached, at 414. The calling subsystem may identify an end of the callable region in the sequencing data when the sequencing data reaches the callable region depth threshold. For example, the callable region depth threshold may be a value of zero or one. When the end of the callable region is identified, the calling subsystem may send the callable region downstream, at 416, to the genotyping subsystem for performing variant calling at 418. If the end of the callable region has not been reached, the calling subsystem may continue to receive sequencing data at 415 and continue monitoring the memory used for storing the callable region at 406 and/or monitoring for an end of the callable region.

If the calling subsystem determines, at 408, that the memory threshold has been reached, then the calling subsystem may determine to spill the callable region to disk at 410. The entirety of the callable region may be spilled to disk, or a portion of the callable region may be maintained in RAM. For example, the portion of the callable region that has been buffered prior to the memory threshold being reached at 408 may be maintained in RAM and sent downstream to the genotyping subsystem for processing. The calling subsystem may send the portion of the callable region that is maintained in RAM downstream to the genotyping subsystem by updating pointers in the buffer to maintain the portion of the callable region in RAM. The spilled portion of the callable region may be compressed and/or stored to the disk.

The genotyping subsystem may stream the callable region from disk at 412. For example, the genotyping subsystem may stream the entire callable region, or a portion thereof, to RAM from disk at 412. The streaming of the callable region may include decompressing the callable region. The genotyping subsystem may stream a portion of the callable region from disk to RAM and analyze the portion of the callable region to determine whether to discard one or more portions of the callable region to free up additional RAM that may not be used for variant calling. For example, the genotyping subsystem may analyze each of the reads in a region. If the reads are equal to the reference, then the reads may be determined not to include a variant and may be discarded or ignored. The genotyping subsystem may also identify and discard or ignore sequencing errors. The genotyping subsystem may monitor another memory threshold while streaming the callable region from disk to ensure that the memory allocation in RAM for the genotyping subsystem is not exceeded while streaming the callable region from disk. The genotyping subsystem may perform variant calling and/or genotyping using the callable region, or portions thereof, uploaded into RAM. One or more portions of the callable region may be used for performing variant calling at 418.

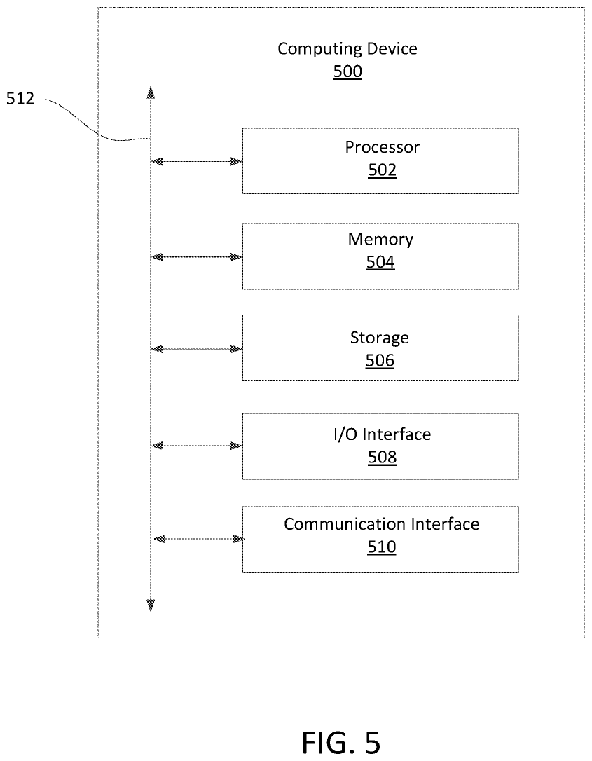

FIG. 5 is a block diagram illustrating an example computing device 500. One or more computing devices such as the computing device 500 may implement one or more features for generating and/or processing callable regions, as described herein. For example, the computing device 500 may comprise one or more of the genotyping device 114, the client device 108, and/or the server device(s) 102 shown in FIG. 1A. As shown by FIG. 5, the computing device 500 may comprise a processor 502, a memory 504, a storage device 506, an I/O interface 508, and/or a communication interface 510, which may be communicatively coupled by way of a communication infrastructure 512. It should be appreciated that the computing device 500 may include fewer or more components than those shown in FIG. 5.

The processor 502 may include hardware for executing instructions, such as those making up a computer program. In examples, to execute instructions for dynamically modifying workflows, the processor 502 may retrieve (or fetch) the instructions from an internal register, an internal cache, the memory 504, or the storage device 506 and decode and execute the instructions. The memory 504 may be a volatile or non-volatile memory used for storing data, metadata, computer-readable or machine-readable instructions, and/or programs for execution by the processor(s) for operating as described herein. The memory 504 may include RAM. The storage device 506 may include storage, such as a hard disk, flash disk drive, or other digital storage device, for storing data or instructions for performing the methods described herein.

The I/O interface 508 may allow a user to provide input to, receive output from, and/or otherwise transfer data to and receive data from the computing device 500. The I/O interface 508 may include a mouse, a keypad or a keyboard, a touch screen, a camera, an optical scanner, network interface, modem, other known I/O devices or a combination of such I/O interfaces. The I/O interface 508 may include one or more devices for presenting output to a user, including, but not limited to, a graphics engine, a display (e.g., a display screen), one or more output drivers (e.g., display drivers), one or more audio speakers, and one or more audio drivers. The L/O interface 508 may be configured to provide graphical data to a display for presentation to a user. The graphical data may be representative of one or more graphical user interfaces and/or any other graphical content.

The communication interface 510 may include hardware, software, or both. In any event, the communication interface 510 may provide one or more interfaces for communication (such as, for example, packet-based communication) between the computing device 500 and one or more other computing devices or networks. The communication may be a wired or wireless communication. As an example, and not by way of limitation, the communication interface 510 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 510 may facilitate communications with various types of wired or wireless networks. The communication interface 510 may also facilitate communications using various communication protocols. The communication infrastructure 512 may also include hardware, software, or both that couples components of the computing device 500 to each other. For example, the communication interface 510 may use one or more networks and/or protocols to enable a plurality of computing devices connected by a particular infrastructure to communicate with each other to perform one or more aspects of the processes described herein. To illustrate, the sequencing process may allow a plurality of devices (e.g., a client device, sequencing device, and server device(s)) to exchange information such as sequencing data and error notifications.

In addition to what has been described herein, the methods and systems may also be implemented in a computer program(s), software, or firmware incorporated in one or more computer-readable media for execution by a computer(s) or processor(s), for example. Examples of computer-readable media include electronic signals (transmitted over wired or wireless connections) and tangible/non-transitory computer-readable storage media. Examples of tangible/non-transitory computer-readable storage media include, but are not limited to, a read only memory (ROM), a random-access memory (RAM), removable disks, and optical media such as CD-ROM disks, and digital versatile disks (DVDs).

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure.

### Clauses

1. A computer-implemented method comprising:
   receiving, at a calling subsystem of a variant caller, sequencing data comprising a plurality of reads of a genome sequence, wherein the calling subsystem of the variant caller is configured to detect a callable region of the sequencing data when a depth of the plurality of reads is above a callable region depth threshold, and wherein the calling subsystem of the variant caller is configured to send at least a portion of the callable region to a genotyping subsystem of the variant caller for variant calling of the callable region;
   monitoring memory used by the calling subsystem of the variant caller;
   when the memory used by the calling subsystem of the variant caller exceeds a memory threshold of a total amount of memory allocated to the calling subsystem of the variant caller, splitting the callable region; and sending a split portion of the callable region to the genotyping subsystem of the variant caller for the variant calling based on the split portion.
2. The computer-implemented method of clause 1, wherein the sending of the split portion of the callable region to the genotyping subsystem increases availability of the memory used by the calling subsystem.
3. The computer-implemented method of clause 1, wherein the memory threshold is a fixed threshold.
4. The computer-implemented method of clause 1, wherein the memory threshold is a dynamic threshold.
5. The computer-implemented method of clause 1, the method further comprising:
   analyzing, at the calling subsystem of the variant caller, the sequencing data when the memory used by the calling subsystem of the variant caller is within the memory threshold of the total amount of memory allocated to the calling subsystem of the variant caller;
   identifying a variant or mutation in the sequencing data within a predefined proximity of an identified split; and
   after identifying the variant or mutation in the sequencing data, performing the splitting of the callable region outside of the predefined proximity of the variant or mutation.
6. The computer-implemented method of clause 5, wherein at least one of the variant or mutation or the predefined proximity of the identified split is determined based on population data that is accessed by the calling subsystem.
7. The computer-implemented method of clause 5, wherein the variant or mutation comprises an insertion or a deletion.
8. The computer-implemented method of clause 1, further comprising:
   analyzing buffered sequencing data to identify a location for the splitting of the callable region within the buffered sequencing data.
9. The computer-implemented method of clause 8, further comprising:
   identifying a portion of the buffered sequencing data having a read depth that is below a splitting threshold; and
   performing the splitting of the buffered sequencing data at the identified portion having the read depth that is below the splitting threshold.
10. The computer-implemented method of clause 1, wherein the callable region is sent to the genotyping subsystem when the depth of the plurality of reads is below the callable region depth threshold that is used to detect the callable region.
11. The computer-implemented method of clause 1, wherein the split portion of the callable region is an entirety of the callable region that is currently in the memory used by the calling subsystem.
12. The computer-implemented method of clause 1, wherein the split portion is a first split portion, the method further comprising:
   maintaining, in the memory used by the calling subsystem of the variant caller, a predefined amount of the sequencing data in the first split portion of the callable region;
   detecting a second split portion of the callable region based on the callable region depth threshold or the memory threshold; and
   sending the second split portion of the callable region to the genotyping subsystem of the variant caller for the variant calling based on the second split portion, wherein the second split portion comprises an overlap in the sequencing data with the first split portion of the callable region that comprises the predefined amount of sequencing data.
13. The computer-implemented method of clause 12, wherein the predefined amount of sequencing data of the overlap is a predefined number of bases.
14. The computer-implemented method of clause 12, wherein the predefined amount of sequencing data of the overlap is determined based on population data that is accessed by the calling subsystem.
15. The computer-implemented method of clause 12, wherein the predefined amount of sequencing data of the overlap is determined based on user input.
16. The computer-implemented method of clause 12, further comprising:
   identifying, at the genotyping subsystem of the variant caller, the overlap in the sequencing data between the first split portion and the second split portion of the callable region; and
   removing the overlap in the sequencing data between the first split portion and the second split portion of the callable region prior to performing the variant calling on the callable region.
17. The computer-implemented method of clause 1, wherein the callable region depth threshold is a first callable region depth threshold, wherein the split portion is a first split portion, and the method further comprising:
   monitoring the depth of the plurality of reads at the calling subsystem of the variant caller;
   when the depth of the plurality of reads is below a second callable region depth threshold, splitting the callable region; and
   sending a second split portion of the callable region to the genotyping subsystem of the variant caller for the variant calling based on the second split portion.
18. A sequencing system comprising:
   memory; and
   at least one processor configured to:
      receive, at a calling subsystem of a variant caller, sequencing data comprising a plurality of reads of a genome sequence, wherein the calling subsystem of the variant caller is configured to detect a callable region of the sequencing data when a depth of the plurality of reads is above a callable region depth threshold, and wherein the calling subsystem of the variant caller is configured to send at least a portion of the callable region to a genotyping subsystem of the variant caller for variant calling of the callable region;
      monitor the memory used by the calling subsystem of the variant caller;
      when the memory used by the calling subsystem of the variant caller exceeds a memory threshold of a total amount of memory allocated to the calling subsystem of the variant caller, split the callable region; and send a split portion of the callable region to the genotyping subsystem of the variant caller for the variant calling based on the split portion.
   19. The sequencing system of clause 18, wherein the at least one processor is configured to send of the split portion of the callable region to the genotyping subsystem to increase availability of the memory used by the calling subsystem.
   20. The sequencing system of clause 18, wherein the memory threshold is a fixed threshold.
   21. The sequencing system of clause 18, wherein the memory threshold is a dynamic threshold.
   22. The sequencing system of clause 18, at least one processor being configured to:
      analyze, at the calling subsystem of the variant caller, the sequencing data when the memory used by the calling subsystem of the variant caller is within the memory threshold of the total amount of memory allocated to the calling subsystem of the variant caller;
      identify a variant or mutation in the sequencing data within a predefined proximity of an identified split; and
      after identifying the variant or mutation in the sequencing data, perform the splitting of the callable region outside of the predefined proximity of the variant or mutation.
   23. The sequencing system of clause 22, wherein at least one of the variant or mutation or the predefined proximity of the identified split is determined based on population data that is accessed by the calling subsystem.
   24. The sequencing system of clause 22, wherein the variant or mutation comprises an insertion or a deletion.
   25. The sequencing system of clause 18, wherein the at least one processor is further configured to:
      analyze buffered sequencing data to identify a location for the splitting of the callable region within the buffered sequencing data.
   26. The sequencing system of clause 25, wherein the at least one processor is further configured to:
      identify a portion of the buffered sequencing data having a read depth that is below a splitting threshold; and
      perform the splitting of the buffered sequencing data at the identified portion having the read depth that is below the splitting threshold.
   27. The sequencing system of clause 18, wherein the at least one processor is configured to send the callable region to the genotyping subsystem when the depth of the plurality of reads is below the callable region depth threshold that is used to detect the callable region.
   28. The sequencing system of clause 18, wherein the split portion of the callable region is an entirety of the callable region that is currently in the memory used by the calling subsystem.
   29. The sequencing system of clause 18, wherein the split portion is a first split portion, and wherein the at least one processor is further configured to:
      maintain, in the memory used by the calling subsystem of the variant caller, a predefined amount of the sequencing data in the first split portion of the callable region;
      detect a second split portion of the callable region based on the callable region depth threshold or the memory threshold; and
      send the second split portion of the callable region to the genotyping subsystem of the variant caller for the variant calling based on the second split portion, wherein the second split portion comprises an overlap in the sequencing data with the first split portion of the callable region that comprises the predefined amount of sequencing data.
   30. The sequencing system of clause 29, wherein the predefined amount of sequencing data of the overlap is a predefined number of bases.
   31. The sequencing system of clause 29, wherein the predefined amount of sequencing data of the overlap is determined based on population data that is accessed by the calling subsystem.
   32. The sequencing system of clause 29, wherein the predefined amount of sequencing data of the overlap is determined based on user input.
   33. The sequencing system of clause 29, wherein the at least one processor is further configured to:
      identify, at the genotyping subsystem of the variant caller, the overlap in the sequencing data between the first split portion and the second split portion of the callable region; and
      remove the overlap in the sequencing data between the first split portion and the second split portion of the callable region prior to performing the variant calling on the callable region.
   34. The sequencing system of clause 18, wherein the callable region depth threshold is a first callable region depth threshold, wherein the split portion is a first split portion, and wherein the at least one processor is further configured to:
      monitor the depth of the plurality of reads at the calling subsystem of the variant caller;
      when the depth of the plurality of reads is below a second callable region depth threshold, split the callable region; and
      send a second split portion of the callable region to the genotyping subsystem of the variant caller for the variant calling based on the second split portion.
   35. At least one computer-readable storage medium having computer-executable instructions stored thereon that, when executed by at least one processor, cause the at least one processor to:
      receive, at a calling subsystem of a variant caller, sequencing data comprising a plurality of reads of a genome sequence, wherein the calling subsystem of the variant caller is configured to detect a callable region of the sequencing data when a depth of the plurality of reads is above a callable region depth threshold, and wherein the calling subsystem of the variant caller is configured to send at least a portion of the callable region to a genotyping subsystem of the variant caller for variant calling of the callable region;
      monitor memory used by the calling subsystem of the variant caller;
      when the memory used by the calling subsystem of the variant caller exceeds a memory threshold of a total amount of memory allocated to the calling subsystem of the variant caller, split the callable region; and
      send a split portion of the callable region to the genotyping subsystem of the variant caller for the variant calling based on the split portion.
   36. The at least one computer-readable storage medium of clause 35, wherein the computer-executable instructions are configured to cause the at least one processor to send of the split portion of the callable region to the genotyping subsystem to increase availability of the memory used by the calling subsystem.
   37. The at least one computer-readable storage medium of clause 35, wherein the memory threshold is a fixed threshold.
   38. The at least one computer-readable storage medium of clause 35, wherein the memory threshold is a dynamic threshold.
   39. The at least one computer-readable storage medium of clause 35, wherein the computer-executable instructions are configured to cause the at least one processor to:
      analyze, at the calling subsystem of the variant caller, the sequencing data when the memory used by the calling subsystem of the variant caller is within the memory threshold of the total amount of memory allocated to the calling subsystem of the variant caller;
      identify a variant or mutation in the sequencing data within a predefined proximity of an identified split; and
      after identifying the variant or mutation in the sequencing data, perform the splitting of the callable region outside of the predefined proximity of the variant or mutation.
   40. The at least one computer-readable storage medium of clause 39, wherein at least one of the variant or mutation or the predefined proximity of the identified split is determined based on population data that is accessed by the calling subsystem.
   41. The at least one computer-readable storage medium of clause 39, wherein the variant or mutation comprises an insertion or a deletion.
   42. The at least one computer-readable storage medium of clause 35, wherein the computer-executable instructions are configured to cause the at least one processor to:
      analyze buffered sequencing data to identify a location for the splitting of the callable region within the buffered sequencing data.
   43. The at least one computer-readable storage medium of clause 42, wherein the computer-executable instructions are configured to cause the at least one processor to:
      identify a portion of the buffered sequencing data having a read depth that is below a splitting threshold; and
      perform the splitting of the buffered sequencing data at the identified portion having the read depth that is below the splitting threshold.
   44. The at least one computer-readable storage medium of clause 35, wherein the computer-executable instructions are configured to cause the at least one processor to send the callable region to the genotyping subsystem when the depth of the plurality of reads is below the callable region depth threshold that is used to detect the callable region.
   45. The at least one computer-readable storage medium of clause 35, wherein the split portion of the callable region is an entirety of the callable region that is currently in the memory used by the calling subsystem.
   46. The at least one computer-readable storage medium of clause 35, wherein the split portion is a first split portion, and wherein the computer-executable instructions are configured to cause the at least one processor to:
      maintain, in the memory used by the calling subsystem of the variant caller, a predefined amount of the sequencing data in the first split portion of the callable region;
      detect a second split portion of the callable region based on the callable region depth threshold or the memory threshold; and
      send the second split portion of the callable region to the genotyping subsystem of the variant caller for the variant calling based on the second split portion, wherein the second split portion comprises an overlap in the sequencing data with the first split portion of the callable region that comprises the predefined amount of sequencing data.
   47. The at least one computer-readable storage medium of clause 46, wherein the predefined amount of sequencing data of the overlap is a predefined number of bases.
   48. The at least one computer-readable storage medium of clause 46, wherein the predefined amount of sequencing data of the overlap is determined based on population data that is accessed by the calling subsystem.
   49. The at least one computer-readable storage medium of clause 46, wherein the predefined amount of sequencing data of the overlap is determined based on user input.
   50. The at least one computer-readable storage medium of clause 46, wherein the computer-executable instructions are configured to cause the at least one processor to:
      identify, at the genotyping subsystem of the variant caller, the overlap in the sequencing data between the first split portion and the second split portion of the callable region; and
      remove the overlap in the sequencing data between the first split portion and the second split portion of the callable region prior to performing the variant calling on the callable region.
   51. The at least one computer-readable storage medium of clause 35, wherein the callable region depth threshold is a first callable region depth threshold, wherein the split portion is a first split portion, and wherein the computer-executable instructions are configured to cause the at least one processor to:
      monitor the depth of the plurality of reads at the calling subsystem of the variant caller;
      when the depth of the plurality of reads is below a second callable region depth threshold, split the callable region; and
      send a second split portion of the callable region to the genotyping subsystem of the variant caller for the variant calling based on the second split portion.
   52. A computer-implemented method comprising:
      receiving, at a calling subsystem of a variant caller, sequencing data comprising a plurality of reads of a genomic sequence, wherein the calling subsystem of the variant caller is configured to detect a callable region of the sequencing data when a depth of the plurality of reads is below a depth threshold, and wherein the calling subsystem of the variant caller is configured to send at least a portion of the callable region to a genotyping subsystem of the variant caller for variant calling of the callable region;
      monitoring memory used by the calling subsystem of the variant caller;
      when the memory used by the calling subsystem of the variant caller is within a memory threshold of a total amount of memory allocated to the calling subsystem of the variant caller, spilling the callable region to disk; and streaming the spilled callable region back from the disk to the memory used by the genotyping subsystem for processing.
   53. The computer-implemented method of clause 52, wherein spilling the callable region to the disk comprises spilling a first portion of the callable region to disk and maintaining a second portion of the callable region in the memory.
   54. The computer-implemented method of clause 53, wherein the second portion of the callable region is sent to the genotyping subsystem for processing via pointers in the memory to maintain the second portion of the callable region in the memory, and wherein the first portion of the callable region is streamed from the disk.
   55. The computer-implemented method of clause 53, further comprising:
      analyzing the spilled callable region that is streamed back from the disk;
      discarding one or more portions of the spilled callable region from the memory that will not be used for the variant calling prior to streaming additional portions of the spilled callable region to the memory.
   56. The computer-implemented method of clause 53, wherein the memory threshold is a first memory threshold associated with the calling subsystem, the method further comprising:
      monitoring a second memory threshold associated with the genotyping subsystem; and
      preventing the second memory threshold from being exceeded while streaming the spilled callable region from the disk.
   57. A sequencing system, comprising:
      memory; and
      at least one processor configured to:
         receive, at a calling subsystem of a variant caller, sequencing data comprising a plurality of reads of a genomic sequence, wherein the calling subsystem of the variant caller is configured to detect a callable region of the sequencing data when a depth of the plurality of reads is below a depth threshold, and wherein the calling subsystem of the variant caller is configured to send at least a portion of the callable region to a genotyping subsystem of the variant caller for variant calling of the callable region;
         monitor the memory used by the calling subsystem of the variant caller;
         when the memory used by the calling subsystem of the variant caller is within a memory threshold of a total amount of memory allocated to the calling subsystem of the variant caller, spill the callable region to disk; and
         stream the spilled callable region back from the disk to the memory used by the genotyping subsystem for processing.
   58. The sequencing system of clause 57, wherein the at least one processor is configured to spill the callable region to the disk by spilling a first portion of the callable region to disk and is further configured to maintain a second portion of the callable region in the memory.
   59. The sequencing system of clause 58, wherein the at least one processor is further configured to send the second portion of the callable region to the genotyping subsystem for processing via pointers in the memory to maintain the second portion of the callable region in the memory, and wherein the at least one processor is further configured to stream the first portion of the callable region from the disk.
   60. The sequencing system of clause 58, wherein the at least one processor is further configured to:
      analyze the spilled callable region that is streamed back from the disk;
      discard one or more portions of the spilled callable region from the memory that will not be used for the variant calling prior to streaming additional portions of the spilled callable region to the memory.
   61. The sequencing system of clause 58, wherein the memory threshold is a first memory threshold associated with the calling subsystem, the at least one processor being further configured to:
      monitor a second memory threshold associated with the genotyping subsystem; and
      prevent the second memory threshold from being exceeded while streaming the spilled callable region from the disk.
   62. At least one computer-readable storage medium having computer-executable instructions stored thereon that, when executed by at least one processor, cause the at least one processor to:
      receive, at a calling subsystem of a variant caller, sequencing data comprising a plurality of reads of a genomic sequence, wherein the calling subsystem of the variant caller is configured to detect a callable region of the sequencing data when a depth of the plurality of reads is below a depth threshold, and wherein the calling subsystem of the variant caller is configured to send at least a portion of the callable region to a genotyping subsystem of the variant caller for variant calling of the callable region;
      monitor memory used by the calling subsystem of the variant caller;
      when the memory used by the calling subsystem of the variant caller is within a memory threshold of a total amount of memory allocated to the calling subsystem of the variant caller, spill the callable region to disk; and
      stream the spilled callable region back from the disk to the memory used by the genotyping subsystem for processing.
   63. The at least one computer-readable storage medium of clause 62, wherein the computer-executable instructions are configured to cause the at least one to spill the callable region to the disk by spilling a first portion of the callable region to disk and is further configured to maintain a second portion of the callable region in the memory.
   64. The at least one computer-readable storage medium of clause 63, wherein the computer-executable instructions are configured to cause the at least one processor to send the second portion of the callable region to the genotyping subsystem for processing via pointers in the memory to maintain the second portion of the callable region in the memory, and wherein the at least one processor is further configured to stream the first portion of the callable region from the disk.
   65. The at least one computer-readable storage medium of clause 63, wherein the computer-executable instructions are configured to cause the at least one processor to:
      analyze the spilled callable region that is streamed back from the disk;
      discard one or more portions of the spilled callable region from the memory that will not be used for the variant calling prior to streaming additional portions of the spilled callable region to the memory.
   66. The at least one computer-readable storage medium of clause 63, wherein the memory threshold is a first memory threshold associated with the calling subsystem, wherein the computer-executable instructions are configured to cause the at least one processor to:
      monitor a second memory threshold associated with the genotyping subsystem; and
      prevent the second memory threshold from being exceeded while streaming the spilled callable region from the disk.

What is claimed is:

1. A computer-implemented method comprising:
receiving, at a calling subsystem of a variant caller, sequencing data comprising a plurality of reads of a genome sequence, wherein the calling subsystem of the variant caller is configured to detect a callable region of the sequencing data when a depth of the plurality of reads is above a callable region depth threshold, and wherein the calling subsystem of the variant caller is configured to send at least a portion of the callable region to a genotyping subsystem of the variant caller for variant calling of the callable region;
monitoring memory used by the calling subsystem of the variant caller;
when the memory used by the calling subsystem of the variant caller exceeds a memory threshold of a total amount of memory allocated to the calling subsystem of the variant caller, splitting the callable region; and
sending a split portion of the callable region to the genotyping subsystem of the variant caller for the variant calling based on the split portion.

2. The computer-implemented method of claim 1, wherein the sending of the split portion of the callable region to the genotyping subsystem increases availability of the memory used by the calling subsystem.

3. The computer-implemented method of claim 1, wherein the memory threshold is a fixed threshold.

4. The computer-implemented method of claim 1, wherein the memory threshold is a dynamic threshold.

5. The computer-implemented method of claim 1, the method further comprising:
analyzing, at the calling subsystem of the variant caller, the sequencing data when the memory used by the calling subsystem of the variant caller is within the memory threshold of the total amount of memory allocated to the calling subsystem of the variant caller;
identifying a variant or mutation in the sequencing data within a predefined proximity of an identified split; and
after identifying the variant or mutation in the sequencing data, performing the splitting of the callable region outside of the predefined proximity of the variant or mutation.

6. The computer-implemented method of claim 5, wherein at least one of the variant or mutation or the predefined proximity of the identified split is determined based on population data that is accessed by the calling subsystem.

7. The computer-implemented method of claim 5, wherein the variant or mutation comprises an insertion or a deletion.

8. The computer-implemented method of claim 1, further comprising:
analyzing buffered sequencing data to identify a location for the splitting of the callable region within the buffered sequencing data.

9. The computer-implemented method of claim 8, further comprising:
identifying a portion of the buffered sequencing data having a read depth that is below a splitting threshold; and
performing the splitting of the buffered sequencing data at the identified portion having the read depth that is below the splitting threshold.

10. The computer-implemented method of claim 1, wherein the callable region is sent to the genotyping subsystem when the depth of the plurality of reads is below the callable region depth threshold that is used to detect the callable region.

11. The computer-implemented method of claim 1, wherein the split portion of the callable region is an entirety of the callable region that is currently in the memory used by the calling subsystem.

12. The computer-implemented method of claim 1, wherein the split portion is a first split portion, the method further comprising:
maintaining, in the memory used by the calling subsystem of the variant caller, a predefined amount of the sequencing data in the first split portion of the callable region;
detecting a second split portion of the callable region based on the callable region depth threshold or the memory threshold; and
sending the second split portion of the callable region to the genotyping subsystem of the variant caller for the variant calling based on the second split portion, wherein the second split portion comprises an overlap in the sequencing data with the first split portion of the callable region that comprises the predefined amount of sequencing data.

13. The computer-implemented method of claim 12, wherein the predefined amount of sequencing data of the overlap is a predefined number of bases.

14. The computer-implemented method of claim 12, wherein the predefined amount of sequencing data of the overlap is determined based on population data that is accessed by the calling subsystem.

15. The computer-implemented method of claim 12, wherein the predefined amount of sequencing data of the overlap is determined based on user input.

16. The computer-implemented method of claim 12, further comprising:
identifying, at the genotyping subsystem of the variant caller, the overlap in the sequencing data between the first split portion and the second split portion of the callable region; and
removing the overlap in the sequencing data between the first split portion and the second split portion of the callable region prior to performing the variant calling on the callable region.

17. The computer-implemented method of claim 1, wherein the callable region depth threshold is a first callable region depth threshold, wherein the split portion is a first split portion, and the method further comprising:
monitoring the depth of the plurality of reads at the calling subsystem of the variant caller;
when the depth of the plurality of reads is below a second callable region depth threshold, splitting the callable region; and
sending a second split portion of the callable region to the genotyping subsystem of the variant caller for the variant calling based on the second split portion.

18. A computer-implemented method comprising:
receiving, at a calling subsystem of a variant caller, sequencing data comprising a plurality of reads of a genomic sequence, wherein the calling subsystem of the variant caller is configured to detect a callable region of the sequencing data when a depth of the plurality of reads is below a depth threshold, and wherein the calling subsystem of the variant caller is configured to send at least a portion of the callable region to a genotyping subsystem of the variant caller for variant calling of the callable region;
monitoring memory used by the calling subsystem of the variant caller;
when the memory used by the calling subsystem of the variant caller is within a memory threshold of a total amount of memory allocated to the calling subsystem of the variant caller, spilling the callable region to disk; and
streaming the spilled callable region back from the disk to the memory used by the genotyping subsystem for processing.

19. The computer-implemented method of claim 18, wherein spilling the callable region to the disk comprises spilling a first portion of the callable region to disk and maintaining a second portion of the callable region in the memory.

20. The computer-implemented method of claim 19, wherein the second portion of the callable region is sent to the genotyping subsystem for processing via pointers in the memory to maintain the second portion of the callable region in the memory, and wherein the first portion of the callable region is streamed from the disk.

21. The computer-implemented method of claim 19, further comprising:
analyzing the spilled callable region that is streamed back from the disk;
discarding one or more portions of the spilled callable region from the memory that will not be used for the variant calling prior to streaming additional portions of the spilled callable region to the memory.

22. The computer-implemented method of claim 19, wherein the memory threshold is a first memory threshold associated with the calling subsystem, the method further comprising:
monitoring a second memory threshold associated with the genotyping subsystem; and
preventing the second memory threshold from being exceeded while streaming the spilled callable region from the disk.
